(19) United States
(12) Patent Application Publication
(10) Pub. No.: US 2026/0000072 A1
(43) Pub. Date: Jan. 1, 2026

(54) SELF-LUBRICATING COMPOSITIONS

(71) Applicant: SLIBIO LABS LTD, Ma'ale Eron (IL)

(72) Inventor: Mahmood AGBARIA, Ma'ale Eron (IL)

(73) Assignee: SLIBIO LABS LTD, Ma'ale Eron (IL)

(21) Appl. No.: 19/164,328

(22) PCT Filed: Mar. 14, 2024
(86) PCT No.: PCT/IL2024/050273

§ 371 Date: Sep. 11, 2025

(30) Foreign Application Priority Data
Mar. 16, 2023 (IL) 301438

(51) Int. Cl. A01N 25/30; A01N 25/02; A01N 25/28; A01P 17/00

(52) U.S. Cl. CPC A01N 25/30; A01N 25/02; A01N 25/28; A01P 17/00

(57) ABSTRACT

The invention provides a self-lubricating composition and methods of using same for preventing insects from climbing on a climbable surface.

### FIELD OF THE INVENTION

This invention provides a self-lubricating composition and methods of using same for preventing insects from climbing on a climbable surface.

### BACKGROUND OF THE INVENTION

Insects, being the main reason for the spread of contagious diseases around the world, are the main cause of ecological damage. Damages caused by insects to agricultural land and to agricultural products increases annually and reaches more than 77 billion dollars. Methods of eradicating insects/pests typically make use of chemical pesticides which, while effective to some degree, lead to additional ecological and agricultural damages and increase the risk to animal and human wellbeing. With the strong relationship existing between increased global temperatures and the expansion of crop pests, the use of chemical pesticides has increased as well, leading also to the disproportionately high number of resistant insects.

Integrated pest management (IPM) has been introduced as a potential methodology for growing healthy crops while minimizing use of chemical pesticides. The aim of the IPM is to prevent insects from inflicting economic or aesthetic damage with the least risk to the environment. However, IPM typically requires complex and time-consuming efforts which require the identification of insects, accurate measurement of their populations, assessment of the damage levels and determining proper management strategies for increasing and maintaining insect control.

Physical and mechanical controls have been proposed and used-some with a degree of success. Ecological landscaping has been proposed to reduce field size and distance to habitats of natural insects and include erection of barriers, covering of crops, mechanical removal of insects and others.

### SUMMARY OF THE INVENTION

The inventors of the technology disclosed herein have realized that controlling damage caused by insects may be successfully achieved by minimizing the ability of insects to climb a surface bridging or extending their natural habitat and a potentially insect-attracting surface likely to be damaged by the insects. Surfaces coated with a self-lubricating formulation of the invention were found effective barriers to insect crossing or movement.

Formulations of the invention are green formulations which do not cause, directly or indirectly, damage to the environment, to the plant or surface onto which the formulation is applied, to humans or animals present in the environment and are not insecticides or pesticides in any way.

Formulation of the invention may be regarded film-forming formulations. The term “film forming”, when used in connection with formulations of the invention, encompasses a provision of generating a dry film on a surface region, wherein the dry film comprises components of the formulation, as disclosed herein.

In a first of its aspects, the invention provides a formulation, e.g., film-forming formulation, comprising at least one film forming material, a mixture of fatty materials (e.g., oils or fats) having a multiplicity of molecular weights and/or viscosities, and optionally a surfactant.

Further provided is an aqueous formulation comprising at least one film forming material, a mixture of fatty materials (e.g., oils or fats) having a multiplicity of molecular weights and/or viscosities, and optionally a surfactant, wherein the formulation is configured for forming a film or a coating substantially not crossable by an insect.

In some embodiments, the film forming material is provided in a form of capsules or carriers capable of holding or containing some of the mixture of fatty materials and optionally the surfactant. Thus, in some embodiments, the formulation comprises a plurality of causes of the at least one film forming material, wherein some or a portion or an amount of the mixture of fatty materials (e.g., oils or fats), and optionally a surfactant, is contained within the capsules, as further disclosed herein.

In some embodiments, the film forming material is provided in a form of capsules or carriers comprising at least a portion of said mixture of fatty materials and optionally the surfactant, wherein the remaining of said mixture and optionally the surfactant are formulated or provided external to said capsules. In other words, in some embodiments, the formulation may comprise (a) a plurality of capsules comprising said mixture of fatty materials and optionally the surfactant; and (b) a mixture of the fatty materials and optionally the surfactant outside of the capsules. The two populations of fatty materials, namely that contained within the capsules and that present outside of said capsules, may be same or different.

In some embodiments, the formulation may comprise two or more different populations of fatty materials, each being distinguishable in composition and/or amount (concentration, material ratios, etc) and/or form of presentation (i.e., loaded in a carrier or capsule or freely distributed in the formulation). In some embodiments, one population of fatty materials is contained in capsules and another is contained in the formulation but outside or external to the capsules. Similarly, two same or different surfactants may be used; wherein, in some cases one surfactant is contained in the capsules and the other outside of the capsules.

In some embodiments, the capsules are selected from core/shell capsules, microcapsules, microcarriers, microspheres and others.

In some embodiments, the formulation comprises two main material components or parts or compositions: a medium and capsules. The medium may comprise a population of fatty materials (e.g., oils or fats) having a multiplicity of molecular weights and/or viscosities, and the capsules which are carried by the medium and which contain a population of fatty materials which may be same or different from the fatty materials in the medium.

The inventio thus provides a formulation comprising (a) a plurality of capsules, e.g., core/shell capsules, formed of a first film-forming material, wherein the capsules, e.g., core of said capsules, comprising or being formed of a first mixture of fatty materials (e.g., oils or fats) having a first multiplicity of molecular weights and/or viscosities; and (b) a mixture of a second film forming material and a second mixture of fatty materials (e.g., oils or fats) having a second multiplicity of molecular weights and/or viscosities, wherein said second mixture of fatty materials being external to or outside of said capsules, e.g., core/shell capsules. The first mixture and the second mixture may be same or different.

One or more surfactants may also be contained in either the capsules or the medium, or in both. Thus, in some embodiments, the formulation comprises (c) one or more first surfactants.

In some embodiments, the formulation is configured or adapted or intended for coating or for forming a film or a coat on a surface region of an object for preventing insects from crossing the surface. In some embodiments, the surface is a climbable surface and the formulation is intended to prevent insects from climbing on the surface.

The invention further provides a formulation for coating a surface, e.g., a climbable surface, for preventing insects from crossing or climbing said surface, the formulation comprising (a) a plurality of core/shell capsules formed of a first film forming material, wherein the core of said capsules comprising a first mixture of fatty materials (oils or fats) having a first multiplicity of molecular weights and/or viscosities; (b) a mixture of a second film-forming materials and a second mixture of fatty materials (oils or fats) having a second multiplicity of molecular weights and/or viscosities and being external to said core/shell capsules; and (c) a first surfactant and optionally a second or further surfactant, wherein the first mixture and the second mixture may be same or different (i.e., in composition, form, etc), wherein the first surfactant and each of the second or further surfactant is same or different, and wherein the first film forming material and the second film forming material are the same or different.

In some embodiments, the first film-forming material used in formulations of the invention is provided in a particle shape, namely as capsules, while the second film-forming material is not provided shaped but rather solubilized in the medium component. In other words, the film-forming materials, be them the same material or different materials, may be provided in formulations of the invention in two different presentations: a first as capsules, as disclosed herein, and the second as a soluble material, in a non-capsule form. In cases where the formulations are free of capsules, the film-forming material is presented in a non-capsule form.

Formulations of the invention are intended for forming a film or a coat on a surface onto which the presence of an insect is not desired. Despite the fact that the formulations are not insecticidal nor may comprise any insecticidal material or insect-repelling materials, a film of the formulation formed on a surface renders insect interaction with the surface nearly impossible. The diminished ability of insects to interact with the surface greatly reduces the insects' ability to wonder around the surface, and the film or coat is thus “substantially not crossable” or “not crossable” by the insects. This effect is greatly magnified where the surface is an inclined or a vertical surface and where an interaction between the insect's legs (or adhesive pads, claws, suction cups, tiny hairs, wrapping means, and silk threads) and the surface is essential for maintaining a hold of the surface. Without wishing to be bound by theory, it is believed that a film or a coating formed of formulations of the invention dramatically reduces or prevents a possible interaction of the insect's body part used for advancing or climbing (such as legs, adhesive pads, claws, suction cups, tiny hairs, wrapping means, and silk threads) with the surface by interrupting one or more mechanical or chemical/biological mechanism of interaction. These interactions may vary or may be used in concert to increase stability. The interaction may be or may involve temporary adherence to the surface, van der Waals interactions, suction interactions, lock-and-key interactions, grabbing or hooking interactions, and others.

Generally speaking, insects are equipped with a variety of means for interacting with a surface. For example, insects equipped with adhesive pads use specialized structures on their Tarsus (foot segments) that secrete a sticky substance. This secretion allows the insects to cling onto various surfaces, enabling them to climb vertically or even navigate upside down. The adhesive pads provide a reliable grip, facilitating movement on diverse substrates. Other insects may employ claws and hooks at the tips of their legs to grasp onto surfaces. These structures act like miniature anchors, enabling the insect to latch onto irregular textures such as bark, rocks, or crevices and thus provide stability, preventing the insect from slipping or falling during climbing. Suction cups provided on insect legs can also be utilized to achieve secure adherence to smooth surfaces. These cups create a vacuum seal, enhancing the insect's ability to climb on wet or slippery substrates like leaves or glass. Suction cups offer a secure attachment and are particularly effective in damp conditions. Similarly, fine microscopic hairs (setae), typically present on the legs and body of certain insects, increase the surface area in contact with the surface, enhancing friction and grip. Tiny hairs are effective for climbing on various surfaces, including vertical ones, and are commonly found in insects like beetles and spiders. Insects with long and flexible bodies may employ a wrapping behavior for climbing. This technique involves winding their bodies around a substrate, such as plant stems or branches, to gain a secure grip. Wrapping behavior is often seen in caterpillars and certain larvae, allowing them to ascend and descend with relative ease. Contrary to such insects, spiders use silk threads for climbing. By releasing silk and allowing it to be carried by the wind, spiders can create a “ballooning” effect, enabling them to ascend into the air and disperse to new locations. Additionally, spiders may use silk draglines to descend from elevated positions or to create lifelines for added stability while climbing.

Surprisingly, coatings formed by applying formulations of the invention on a surface, e.g., a climbable surface, interrupt or disrupt the insect's ability to latch on the coating by any of the mechanisms disclosed above, thereby reducing, diminishing or preventing the insect's ability of crossing the coated surface or climbing thereon. This ability is derived from the self-lubricating capabilities of the formulation and the coating or films made therefrom. The composition of the coating or film provides constant lubrication to the (climbable) surface, due to a prolonged release of the fatty materials from the coating or film. Upon application onto the surface, the water carrier evaporates leaving in behind a film or a coat or a layer of the film-forming material embedding the mixture of fatty materials (oils or fats). Where capsules are present in the formulation, upon evaporation of water, a film or a coat is formed that embeds both the capsules containing the fatty mixture and the fatty mixture that is external to the capsules (the so-called second mixture). Continuous release of oils and fats from the film or coat wets the surface over a period of time until the capsules which may be present in the film or coat begin releasing the oils or fats from the so-called first mixture to continuously wet the surface.

The mixture(s) of fatty materials provided in the core/shell capsules (first mixture) or external to said capsules (second mixture) or in formulations not containing capsules may be same or different. Independent of their composition and the formulations used (formulations containing capsules or free of capsules), the mixtures are provided in “a multiplicity of molecular weights and/or viscosities”, namely in a mixture comprising fatty materials having different molecular weights and/or different viscosities. In addition to the sequenced release described above, the release or diffusion of the oils or fats of different molecular weights or viscosities is at a rate that may be directly proportional to the material molecular weight and/or its viscosity. Thus, the mixture of fatty materials of different molecular weights and/or viscosities provides an additional sequence of multiple release/diffusion rates or profiles, such that the coating exhibits continued and prolonged lubrication. Typically, the greater the multiplicity or the variability in molecular weights or viscosities, the better the efficiency and/or the time period over which the coating will remain efficient. In other words, the greater the number of materials (oils or fats) present in the first and/or second mixtures that are provided in different molecular weights and/or viscosities, the longer the release is. Low molecular weight/viscosity materials may be released first, followed by release of materials having continuously increasing molecular weights/viscosities.

In some embodiments, the first and second mixtures, independently, are formulated based on a selection of oils or fats having different molecular weights. In some embodiments, the first and second mixtures, independently, are formulated based on a selection of oils or fats having different viscosities. In some embodiments, the first and second mixtures, independently, are formulated based on a selection of oils or fats having different molecular weight and viscosities.

In some embodiments, the first and second mixtures, independently, are formulated based on a selection of oils or fats having different molecular weights ranging between 200 and 200,000 Da. In some embodiments, the first and second mixtures, independently, are formulated based on a selection of oils or fats having different viscosities ranging between 20 and 200,000 cp. In some embodiments, the materials are selected to having a molecular weight between 200 and 200,000 Da and a viscosity between 20 and 200,000 cp.

In some embodiments, the first and second mixtures, independently are formulated based on a selection of oils or fats that are solids or liquids at room temperature (23-33° C.). Thus, in some cases, where a fatty material is a solid at room temperature, it may be selected based on its molecular weight, rather than its viscosity. Materials which are liquids may be selected based on their molecular weights or viscosities or a combination of both.

In some embodiments, the first mixture and second mixture are the same, or have the same profile (namely, composition, amounts, ratio amounts etc). In some embodiments, the first mixture and the second mixture are different in composition, and/or material amounts, and/or material ratios, etc.

The “mixture of fatty materials” is a mixture of two or more oils and/or fats, namely-a mixture or two or more oils, two or more fats, or a mixture of oils and fats or any other fatty material, wherein at least two of the oils or fats are distinguishable by their molecular weights and/or viscosities. The difference in molecular weights or viscosities may be at least 100 Da or 100 cp. The two or more oils or fats may be a mixture of two, three, four or any other number of such fatty materials or a combination comprising any number of different oils or fats. For example, where silicone oils are used, the silicon oils may be selected by their molecular weight, being between 13,000 Da and 140,000 Da, by their viscosities, being between 350 cp and 100,000 cp or a combination of both. In other words, a mixture of fatty materials having a multiplicity of molecular weights and/or viscosities may comprise silicon oil having a molecular weight of 13,600 Da with silicon oils having molecular weights of 116,500 Da and 139,000 Da. Similarly, a mixture of silicon oils may comprise silicon oil having a viscosity of 350 cp with silicon oils having viscosities of 60,000 cp and 10 cp.

Molecular weights and viscosities of a variety of oils and fats are known or may be determined by conventional methods of analysis, including GC-MS, GPC, viscometers and others.

The fatty materials may be selected amongst any oil or fat, as known in the art, wherein the materials are further selected to have a hydrophilic-lipophilic balance (HLB) of 5 or higher.

While the oils and fats selected and used in accordance with the invention may have similar properties, the difference between the two groups of materials may reside in their physical presentation. Fats are typically solid at room temperatures while oils are liquids at room temperatures. Nevertheless, as used herein, a distinction is not made between fats and oils; any fatty material may be used without further classification. Both groups of materials may be synthetic, semi-synthetic or naturally obtained. In some embodiments, the fatty materials may be selected from silicone oils, fruit oils, vegetable oils, animal oils, fatty acids, fatty acid amides, petroleum oils, mineral oils, and others. Non-limiting examples of fatty materials may include silicone oils such as polydimethylsiloxane (PDMS) oil and silicon oils of different molecular weights and/or viscosities (such as silicon oil 350, silicon oil 60000, silicon oil 10, silicon oil 100000 and others); vegetable oils such as palm oil, soybean oil, rapeseed oil, sunflower oil, peanut oil, cottonseed oil, palm kernel oil, canola oil, soya oil, coconut oil, olive oil, corn oil, sesame oil, and others; fruit oils such as almond oil (sweet and/or bitter), grapefruit oil, coconut oil, peach oil, lemon oil, orange oil, avocado oil, olive oil, and others; fatty acids such as caprylic acid, capric acid, lauric acid, myristic acid, palmitic acid, stearic acid, arachidic acid, myristoleic acid, palmitoleic acid, sapienic acid, oleic acid, claidic acid, vaccenic acid, linoleic acid, linoelaidic acid, α-linolenic acid, and others; fatty acid amides such as the amide forms of the aforementioned fatty acids, or fatty acid amides such as crucamide, oleamide, ethylene bis-oleamide, stearyl crucamide, oleyl palmitamide or any combination thereof.

In some embodiments, the fatty acid is a fruit oil such as almond oil, e.g., bitter and/or sweet almond oil. In some embodiments, the oil is a coconut oil, a canola oil or an olive oil.

In some embodiments, the fatty materials used in the first mixture and the second mixture are the same or may be selected from the same group of fatty materials, e.g., from vegetable oils, based on their molecular weights or viscosities. All viscosities presented herein are measured at 25° C. In some embodiments, the first mixture and/or the second mixture comprises at least two fatty materials having different viscosities selected between 5 and 100,000 cp (measured at 25° C.). In some embodiments, the different fatty materials may be selected from such having viscosities between 350-60,000 cp, 100-1,000 cp, 40,000-80,000 cp, 35-45 cp, and 5-80 cp. For example, the first mixture may comprise an oil having a viscosity between 100-1000 cp and another oil of a viscosity between 40,000-80,000 cp. In another example, the first or second or both mixtures may comprise a first oil having a viscosity between 35-45 cp, a second oil with a viscosity between 5-80 cp and a third oil with a viscosity between 100-1000 cp.

In some embodiments, one of the first and second mixtures may comprise almond oil with a viscosity of 35-45 cp and the second of the first and second mixtures may comprise a vegetable oil with a viscosity of between 5-80 cp.

In some embodiments, the first and/or second mixtures may comprise almond oil having a viscosity of 35-45 cp.

In some embodiments, the first and/or second mixtures may comprise a vegetable oil having a viscosity between 10-70 cp.

In some embodiments, the first and/or second mixtures may comprise a vegetable oil having a viscosity between 20-60 cp.

In some embodiments, the first and/or second mixtures may comprise a vegetable oil having a viscosity between 30-50 cp.

In some embodiments, the first and/or second mixtures may comprise a vegetable oil having a viscosity between 40-60 cp.

In some embodiments, the first and/or the second mixture may comprise olive oil having a viscosity of between 40-60 cp. In some embodiments, the olive oil has a viscosity between 45-55 cp.

The fatty material may be a synthetic oil and/or a fatty acid.

In some embodiments, the synthetic oil is or comprises polyalphaolefin (PAO) base oils, paraffin oil, polyalkylene glycol (polyethylene glycol, polypropylene glycol, etc) or any combination thereof.

In some embodiments, the first and/or second mixtures may comprise a synthetic oil or a silicone oil. In some embodiments, the first and/or second mixture may comprise a mixture of a synthetic oil and a silicone oil.

In some embodiments, the first and/or second mixtures may comprise a fatty material selected from palmitic acid, stearic acid, oleic acid, linoleic acid, N-acylethanolamine, oleamide, stearamide, erucamide and any combination thereof.

In some embodiment, the first and/or second mixture may comprise a fatty material being a fatty acid, a fatty acid amide or a combination of the two.

In some embodiments, the first mixture comprises one or more silicone oils, or almond oil, or one or more vegetable oil, or one or more fatty acid amide, or one or more synthetic oil, or one or more fatty acids or any combination thereof. In some embodiments, the first mixture comprises a combination of ant two of one or more silicone oils, almond oil, one or more vegetable oil, one or more fatty acid amide, one or more synthetic oil, one or more fatty acids.

In some embodiments, the second mixture comprises at least one synthetic oil selected from polyalphaolefin (PAO) base oils, paraffin oil, polyalkylene glycol or any combination thereof.

In some embodiments, the second mixture comprises a synthetic oil and/or a silicone oil.

In some embodiments, the second mixture comprises a fatty acid selected from palmitic acid, stearic acid, oleic acid, linoleic acid, N-acylethanolamine, oleamide, stearamide, erucamide or any combination thereof.

In some embodiments, the second mixture comprises one or more silicone oils, or almond oil, or one or more vegetable oil, or one or more fatty acid amide, or one or more synthetic oil, or one or more fatty acids or any combination thereof. In some embodiments, the second mixture comprises a combination of ant two of one or more silicone oils, almond oil, one or more vegetable oil, one or more fatty acid amide, one or more synthetic oil, one or more fatty acids.

Formulations of the invention comprise a plurality of core/shell capsules, which cores comprise or consist the first mixture of fatty materials, as defined, and a shell that is formed of a different material, i.e., film-forming material. Typically, the material composition of each core/shell capsule is such that the shell constitutes between about 30 and 50 wt % of the weight of the capsule, while the first mixture of fatty materials contained in the core of the capsule constitutes between 50 and 70 wt % of the total weight of the capsule.

Where silicon oils are present in the first mixture, the amount of the silicone oil may be between 5 and 40 wt % (relative to the total mixture including shell). In some embodiments, the amount of silicone oils (one or more) is between 5 and 40 wt %, or between 5 and 10 wt %, or between 10 and 20 wt %, or between 20 and 30 wt % or between 30 and 40 wt %.

In some embodiments, the first mixture comprises a combination of fatty materials such as between 5 and 40 wt % of silicone oils, between 0 and 10 wt % almond oil, between 0 and 5 wt % of a fatty acid amide, between 1 and 5 wt % of a surfactant and between 5 and 30 wt % of water. In some embodiments, the amount of the silicone oils (one or more) may be between 5 and 40 wt %, 5 and 10 wt %, 10 and 20 wt %, 20 and 30 wt % or between 30 and 40 wt %.

In some embodiments, the first mixture comprises almond oil and a vegetable oil or mixture thereof, wherein the amount of each may be between 5 and 10 wt %, or between 0 and 5 wt % or between 5 and 10 wt %.

The amount of water in the core of the capsules, i.e., in the first mixture, may be between 5 and 30 wt %, between 5 and 10 wt %, between 10 and 20 wt % or between 20 and 30 wt %.

In some embodiments, the capsules may comprise at least one of polyvinyl alcohol, carnauba wax and bees wax; one or more silicone oil having a viscosity between 100 and 1,000 cp and one or more other silicone oil having a viscosity between 40,000 and 80,000 cp; a mixture of almond oil and a vegetable oil; erucamide, oleamide, ethylene bis-oleamide, stearyl erucamide, and oleyl palmitamide; PHMS-g-PEG, PHMS, PEG or any combination thereof; and water.

In some embodiments, the capsules comprise at least one of polyvinyl alcohol, carnauba wax and bees wax in a weight ratio of 0-3:0-3:0-3; one or more silicone oil having a viscosity between 100 and 1,000 cp and one or more other silicone oil having a viscosity between 40,000 and 80,000 cp; a mixture of almond oil and a vegetable oil; erucamide, oleamide, ethylene bis-oleamide, stearyl erucamide, and oleyl palmitamide; PHMS-g-PEG, PHMS, PEG or any combination thereof; and water.

In some embodiments, the capsules comprise between 30 and 50 wt % of at least one of polyvinyl alcohol, carnauba wax and bees wax in a weight ratio of 0-3:0-3:0-3; between 5 and 40 wt % of one or more silicone oil having a viscosity between 100 and 1,000 cp and one or more other silicone oil having a viscosity between 40,000 and 80,000 cp; between 0 and 10 wt % of a mixture of almond oil and a vegetable oil; between 0 and 5 wt % of erucamide, oleamide, ethylene bis-oleamide, stearyl erucamide, and oleyl palmitamide; between 1 and 5 wt % of PHMS-g-PEG, PHMS, PEG or any combination thereof; and between 5 and 30 wt % water.

In some embodiments, the capsules comprise a shell material that constitutes between 30 and 50 wt % or about 40 wt %; and a core comprising the first mixture of silicone oils, wherein at least one silicone oil having a viscosity of 350 cp and at least another silicone oil having a viscosity of 60,000 cp.

In some embodiments, the first mixture comprises a mixture of silicone oils, almond oil, a vegetable oil and a fatty acid amide.

In some embodiments, the first mixture comprises a mixture of silicone oils, wherein at least one silicone oil having a viscosity between 100 and 1,000 cp and another silicone oil having a viscosity between 40,000 and 80,000 cp.

In some embodiments, the first mixture comprises a silicone oil having a viscosity between 100 and 500 cp, or a viscosity between 100 and 250 cp, or a viscosity between 250 and 1,000 cp, or a viscosity between 250 and 500 cp, or a viscosity of 350 cp, or a viscosity of between 500 and 1,000 cp, or a viscosity between 40,000 and 70,000 cp, or a viscosity between 40,000 and 50,000 cp, or a viscosity between 50,000 and 80,000 cp, or a viscosity between 50,000 and 70,000 cp, or a viscosity between 70,000 and 80,000 cp, or a viscosity of 60,000 cp.

In some embodiments, the first mixture comprises two fatty materials, e.g., two silicone oils, at a weight ratio 1-3:0-1. In some embodiments, the two silicon oils are present at a weight ratio 1-2:0-1. In some embodiments, the weight ratio is 0.5-5:0-2. In some embodiments, the weight ratio is 3:1.

In some embodiments, the first mixture comprises a first silicone oil having a viscosity of 350 cp and a second silicone oil having a viscosity of 60,000 cp, present at a weight ratio (first:second oils) that is 1-3:0-1, or 1-2:0-1, or 0.5-5:0-2, or 3:1.

In some embodiments, the first mixture comprises almond oil having a viscosity between 35 and 45 cp. In some embodiment, the first mixture comprises a vegetable oil having a viscosity between 5 and 80 cp, or between 10 and 70 cp, or between 20 and 60 cp, or between 30 and 50 cp, or between 40 and 60 cp.

As stated herein, the first mixture of fatty materials is contained in a core of the capsule, while the second mixture of fatty materials is contained outside of the capsules. Putting it differently, the capsules are contained within or carried by, or suspended in the second mixture of fatty materials. In some embodiments, the second mixture of materials is the same as the first mixture of materials. In other cases, the two mixtures may be different in composition, amounts, material ratios, etc.

In some embodiments, the second mixture may comprise almond oil with a viscosity between 35 and 45 cp and/or a vegetable oil with a viscosity between 5 and 80 cp.

In some embodiments, the first and/or second mixtures may comprise almond oil having a viscosity of 35-45 cp.

In some embodiments, the second mixture may comprise a vegetable oil having a viscosity between 10 and 70 cp, or between 20 and 60 cp, or between 30 and 50 cp, or between 40 and 60 cp.

In some embodiments, the second mixture may comprise olive oil having a viscosity of between 40 and 60 cp and/or a viscosity between 45 and 55 cp.

In some embodiments, the second mixture may comprise a fatty material which may be a synthetic oil and/or a fatty acid. In some embodiment, the synthetic oil is or comprises polyalphaolefin (PAO) base oils, paraffin oil, polyalkylene glycol or any combination thereof.

In some embodiments, the second mixture may comprise a synthetic oil or a silicone oil. In some embodiments, the second mixture may comprise a mixture of a synthetic oil and a silicone oil.

In some embodiments, the second mixture may comprise a fatty material selected from palmitic acid, stearic acid, oleic acid, linoleic acid, N-acylethanolamine, oleamide, stearamide, erucamide and any combination thereof.

In some embodiments, the second mixture may comprise a fatty material being a fatty acid, a fatty acid amide or a combination of the two.

In some embodiments, the second mixture comprises one or more silicone oils, or almond oil, or one or more vegetable oil, or one or more fatty acid amide, or one or more synthetic oil, or one or more fatty acids or any combination thereof. In some embodiments, the second mixture comprises a combination of ant two of one or more silicone oils, almond oil, one or more vegetable oil, one or more fatty acid amide, one or more synthetic oil, one or more fatty acids.

In some embodiments, the second mixture comprises at least one synthetic oil selected from polyalphaolefin (PAO) base oils, paraffin oil, polyalkylene glycol or any combination thereof.

In some embodiments, the second mixture comprises a synthetic oil and/or a silicone oil.

In some embodiments, the second mixture comprises a fatty acid selected from palmitic acid, stearic acid, oleic acid, linoleic acid, N-acylethanolamine, oleamide, stearamide, erucamide or any combination thereof.

In some embodiments, the second mixture comprises one or more silicone oils, or almond oil, or one or more vegetable oil, or one or more fatty acid amide, or one or more synthetic oil, or one or more fatty acids or any combination thereof. In some embodiments, the second mixture comprises a combination of ant two of one or more silicone oils, almond oil, one or more vegetable oil, one or more fatty acid amide, one or more synthetic oil, one or more fatty acids.

In some embodiments, the second mixture may comprise a combination of fatty materials such as between 5 and 40 wt % of silicone oils, between 0 and 10 wt % almond oil, between 0 and 5 wt % of a fatty acid amide, between 1 and 5 wt % of a surfactant and between 5 and 30 wt % of water. In some embodiments, the amount of the silicone oils (one or more) may be between 5 and 40 wt %, 5 and 10 wt %, 10 and 20 wt %, 20 and 30 wt % or between 30 and 40 wt %.

In some embodiments, the second mixture comprises almond oil and a vegetable oil or mixture thereof, wherein the amount of each may be between 5 and 10 wt %, or between 0 and 5 wt % or between 5 and 10 wt %.

The amount of water in the core of the capsules, i.e., in the second mixture, may be between 5 and 30 wt %, between 5 and 10 wt %, between 10 and 20 wt % or between 20 and 30 wt %.

In some embodiments, the second mixture may comprise at least one of polyvinyl alcohol, carnauba wax and bees wax; one or more silicone oil having a viscosity between 100 and 1,000 cp and one or more other silicone oil having a viscosity between 40,000 and 80,000 cp; a mixture of almond oil and a vegetable oil; erucamide, oleamide, ethylene bis-oleamide, stearyl erucamide, and oleyl palmitamide; PHMS-g-PEG, PHMS, PEG or any combination thereof; and water.

In some embodiments, the second mixture may comprise at least one of polyvinyl alcohol, carnauba wax and bees wax in a weight ratio of 0-3:0-3:0-3; one or more silicone oil having a viscosity between 100 and 1,000 cp and one or more other silicone oil having a viscosity between 40,000 and 80,000 cp; a mixture of almond oil and a vegetable oil; erucamide, oleamide, ethylene bis-oleamide, stearyl erucamide, and oleyl palmitamide; PHMS-g-PEG, PHMS, PEG or any combination thereof; and water.

In some embodiments, the second mixture may comprise between 30 and 50 wt % of at least one of polyvinyl alcohol, carnauba wax and bees wax in a weight ratio of 0-3:0-3:0-3; between 5 and 40 wt % of one or more silicone oil having a viscosity between 100 and 1,000 cp and one or more other silicone oil having a viscosity between 40,000 and 80,000 cp; between 0 and 10 wt % of a mixture of almond oil and a vegetable oil; between 0 and 5 wt % of erucamide, oleamide, ethylene bis-oleamide, stearyl erucamide, and oleyl palmitamide; between 1 and 5 wt % of PHMS-g-PEG, PHMS, PEG or any combination thereof; and between 5 and 30 wt % water.

In some embodiments, the second mixture comprises a mixture of silicone oils, almond oil, a vegetable oil and a fatty acid amide.

In some embodiments, the second mixture comprises a mixture of silicone oils, wherein at least one silicone oil having a viscosity between 100 and 1,000 cp and another silicone oil having a viscosity between 40,000 and 80,000 cp.

In some embodiments, the second mixture comprises a silicone oil having a viscosity between 100 and 500 cp, or a viscosity between 100 and 250 cp, or a viscosity between 250 and 1,000 cp, or a viscosity between 250 and 500 cp, or a viscosity of 350 cp, or a viscosity of between 500 and 1,000 cp, or a viscosity between 40,000 and 70,000 cp, or a viscosity between 40,000 and 50,000 cp, or a viscosity between 50,000 and 80,000 cp, or a viscosity between 50,000 and 70,000 cp, or a viscosity between 70,000 and 80,000 cp, or a viscosity of 60,000 cp.

In some embodiments, the second mixture comprises two fatty materials, e.g., two silicone oils, at a weight ratio 1-3:0-1. In some embodiments, the two silicon oils are present at a weight ratio 1-2:0-1. In some embodiments, the weight ratio is 0.5-5:0-2. In some embodiments, the weight ratio is 3:1.

In some embodiments, the second mixture comprises a first silicone oil having a viscosity of 350 cp and a second silicone oil having a viscosity of 60,000 cp, present at a weight ratio (first:second oils) that is 1-3:0-1, or 1-2:0-1, or 0.5-5:0-2, or 3:1.

In some embodiment, the second mixture comprises almond oil having a viscosity between 35 and 45 cp. In some embodiment, the second mixture comprises a vegetable oil having a viscosity between 5 and 80 cp, or between 10 and 70 cp, or between 20 and 60 cp, or between 30 and 50 cp, or between 40 and 60 cp.

Each of the embodiments disclosed herein with regard to the first mixture may be combinable with any of the other embodiments disclosed herein with regard to the second mixture.

In some embodiment, the second mixture of fatty materials may comprise a mixture of silicone oils; almond oil; and a vegetable oil.

In some embodiments, the second mixture may comprise a mixture of silicone oils, wherein at least one of the silicone oils having a viscosity between 100 and 1,000 cp (or between 100 and 500 cp, or between 100 and 250 cp, or between 250 and 1,000 cp, or between 250 and 500 cp, or between 500 and 1,000 cp, or a viscosity that us 350 cp) and a further silicone oil having a viscosity between 40,000 and 80,000 cp (or between 40,000 and 70,000 cp, or between 40,000 and 50,000 cp, or between 50,000 and 80,000 cp, or between 50,000 and 70,000 cp, or between 70,000 and 80,000 cp or a viscosity of 60,000 cp).

In some embodiment, the second mixture comprises a mixture of silicone oils comprising a silicone oil having a viscosity of 350 cp and a silicone oil having a viscosity of 60,000 cp. In some embodiments, the weight ratio of the two silicon oils is 1-3:0-1, or 1-2:0-1, or 0.5-5:0-2, or 3:1.

In some embodiments, the second mixture comprises almond oil having a viscosity between 35 and 45 cp and/or a vegetable oil having a viscosity between 5 and 80 cp, or between 10 and 70 cp, or between 20 and 60 cp, or between 30 and 50 cp or between 40 and 60 cp.

Water may be present in either or both the first and second mixtures.

In some embodiments, in formulations of the invention not containing capsules, the content of the fatty materials, the film forming materials and the surfactant are as defined for the second mixture of fatty materials, second film forming material and second surfactant. In some embodiments, in formulations of the invention not containing capsules, the content of the fatty materials, the film forming materials and the surfactant are as defined for the first mixture of fatty materials, first film forming material and first surfactant.

The first and second mixtures, independently, may comprise one or more surfactants. The surfactant used in the first mixture may be same or different than that used in the second mixture. In some embodiments, one of the mixtures comprises a surfactant. In some embodiments, both mixtures comprise the same or different surfactants.

In some embodiments, the same surfactant is used for both the first and the second mixtures.

The surfactant used may be any such material known in the art. In some embodiments, the surfactant is selected to stabilize the formulation and prevent agglomeration or breaking of the emulsion, dispersion or suspension that characterizes the formation of the invention. Non-limiting examples include PHMS-g-PEG, PHMS, polyethylene glycol (PEG), PDMS and polyether in decamethylcyclopentasiloxane (Dow corning 5225c), Dow corning 5200, RITASIL sw 3050 CETYL PEG-PPG-10/1 DIMETHICONE, ABIL care 85-Bis-PEG/PPG-16/16 PEG/PPG-16/16 Dimethicone (and) Caprylic/Capric Triglyceride, alkylated vinyl pyrrolidone polymers, cyclopentasiloxane, PEG/PPG-18/18 Dimethicone, lauryl PEG/PPG-18/18 methicone, cetyl PEG-PPG-10/1 dimethicone, bis-PEG/PPG-16/16 PEG/PPG-16/16 dimethicone, caprylic/capric triglyceride, 1-butene-N-vinylpyrrolidone copolymer, 1-hexadecnyl-N-vinylpyrrolidone copolymer, or any combination thereof.

In some embodiments, the first and second surfactants are each independently selected from PHMS-g-PEG, PHMS and PEG.

In some embodiments, the first and/or the second surfactants may be an adduct of PHMS and PEG, wherein the adduct is as defined herein. In some embodiments, the surfactant is represented by formula (I) and/or (II), as defined herein.

Formulations of the invention comprise a plurality of capsules which make up, in some embodiments, between about 5 and 30 wt % and a mixture of fatty materials (the so-called second mixture) that constitutes the remaining part of the formulation.

Formulations of the invention are film forming formulations, as defined herein. The “film-forming material” present in the formulations, by itself or in combination with other components of the formulation, is capable of causing the formulation to form a continuous dry film on the surface onto which it is applied. The film-forming material is selected to facilitate application of the formulation, and endows the film with mechanical characteristics that prevent the film from easily peeling off, cracking, or splitting and which can nevertheless effectively hold the components of the formulation and permit their continued release. The film forming material may be used or provided as a solubilized material which can form the continuous film on the surface, and/or may be used for forming the capsules present in some formulations of the invention.

In some embodiments, the film forming material may be a suitable polymer or a wax material. In some embodiments, the film-forming material is a biodegradable material, e.g., a biodegradable polymer. In some embodiments, the film-forming material may be selected from polyvinyl alcohol (PVOH), polyvinyl acetate (PVAc), polycaprolactone (PCL), a fatty alcohol, a biodegradable polyester, poly (furfuryl alcohol) bio-resin, a cellulosic material, lignin, a biobased wax, a paraffin wax, and any combination thereof.

In some embodiments, the formulation of the invention comprises a plurality of capsules, wherein each of the capsules having a shell formed of a film-forming material. In such formulations, same or different film-forming materials may be present as a material in the medium, not part of the capsules shells.

In some embodiments, the formulation of the invention is free of capsules and the film-forming material is used with the fatty materials for forming the coat or film.

As used herein, the term “capsule” relates to a unit comprising a core material, i.e., the second mixture of fatty materials, and a shell of a film-forming material that encircles or encapsulates the core material. The capsule is therefore a core/shell structure. While the capsule may be of any shape and size, it is typically a circular unit having a micrometric size. The capsule may thus be regarded a microcapsule, a microcarrier, or a microsphere. The size (diameter) of the capsule may be between 1 and 1,000μ (micron). In some embodiments, the size of the microcapsule is between 1 and 900, 1 and 800, 1 and 700, 1 and 600, 1 and 500, 1 and 400, 1 and 300, 1 and 200, 1 and 100, 1 and 50, 1 and 10, 1 and 5, 10 and 1,000, 10 and 500, 10 and 100, 10 and 50, 50 and 1,000, 50 and 500, 50 and 100, 100 and 1000, 100 and 500, or between 500 and 1,000μ (microns).

The capsules may be preformed or may be generated during the preparation of the formulation. Notwithstanding the method of capsule preparation, loading of the capsules with a material composition disclosed herein may be achieved in a variety of ways, as known in the art.

The shell of the capsules is typically made of a film-forming material that is a biodegradable material that can degrade under certain conditions, such as mechanical conditions or in presence of water or humidity, to release its content (i.e., the first mixture). The shell material may be a solid or a semi-solid material that maintains its integrity in the formulation and begins dissociation or degradation when exposed to environmental conditions or due to mechanical forces. The shell material, i.e., the film-forming material, may be polymeric or made of a high molecular material. In some embodiments, the shell material is a polymeric material or a wax material, which may be synthetic, semi-synthetic or natural. Non-limiting examples of a shell material include polyvinyl alcohol (PVOH), polyvinyl acetate (PVAc), polycaprolactone (PCL), a fatty alcohol, a biodegradable polyester, poly (furfuryl alcohol) bio-resin, a cellulosic material, lignin, a biobased wax, a paraffin wax, and any combination thereof.

In some embodiments, the shell is or comprises one or more fatty alcohols. The fatty alcohol may be selected from lauryl alcohol, stearyl alcohol, cetyl alcohol, and olcyl alcohols.

In some embodiments, the shell is or comprises one or more biodegradable polyester. The biodegradable polyester may be selected from polylactic acid, polybutylene succinate (PBS), polycaprolactone (PCL), poly (lactic acid)/poly (butylene succinate) (PBSA), polyhydroxybutyrate (PHB), poly (3-hydroxyvalerate) (PHV), polyhydroxy butyrate (PHH), and polybutylene adipate terephthalate (PBAT).

In some embodiments, the shell is or comprises one or more poly (furfuryl alcohol) bio-resin.

In some embodiments, the shell is or comprises a cellulosic material. The cellulosic material may be selected from cellulose, nanocrystalline cellulose (NCC), cellulose acetate and hemicellulose.

In some embodiment, the shell is or comprises a biobased wax. The biobased wax may be selected from carnauba wax, bees wax, shellfish wax, lanolin, candelilla wax, jojoba oil, soy wax and any combination thereof.

In some embodiments, the shell comprises polyvinyl alcohol, carnauba wax and bee wax. In some embodiments, the shell comprises polyvinyl alcohol, carnauba wax and bees wax at a weight ratio of 0-3:0-3:0-3, respectively. In some embodiment, the ratio is 0-2:0-2:0-2, or 0-1:0-1:0-1.

The invention further provides a method of preventing insects from crossing or climbing a surface, the method comprising forming a coating or a film on said surface, wherein said coating or film comprises a film-forming material, a mixture of fatty materials (e.g., oils or fats) having a multiplicity of molecular weights or viscosities, and optionally a surfactant.

In some embodiments, the coating or film comprises any composition as disclosed herein.

In some embodiments, the coating or film is formed by applying onto the surface an aqueous formulation as disclosed herein, e.g., a formulation comprising at least one film forming material, a mixture of fatty materials (e.g., oils or fats) having a multiplicity of molecular weights or viscosities, and optionally a surfactant.

In some embodiments, the film forming material is provided in a form of capsules or carriers at least partially comprising said mixture of fatty materials and optionally the surfactant. In some embodiments, the film forming material is PVOH.

In some embodiments, the film forming material is provided in a form of capsules or carriers comprising at least a portion of said mixture of fatty materials and optionally the surfactant, wherein the remaining of said mixture and optionally the surfactant are formulated or provided external to said capsules. Thus, the formulation may comprise (a) a plurality of capsules comprising said mixture of fatty materials and optionally the surfactant; and (b) a mixture of the fatty materials and optionally the surfactant outside of the capsules.

In some embodiments, the formulation may comprise two or more different populations of fatty materials, each being distinguishable in composition and/or amount (concentration, material ratios, etc) and/or form of presentation (i.e., loaded in a carrier or capsule or freely distributed in the formulation).

In some embodiments, the capsules are selected from core/shell capsules, microcapsules, microcarriers, and others.

In some embodiments, the coat or film comprises (a) a plurality of core/shell capsules formed of a first film-forming material, wherein the core of said capsules comprising or being formed of a first mixture of fatty materials (e.g., oils or fats) having a first multiplicity of molecular weights or viscosities; (b) a mixture of a second film-forming material and a second mixture of fatty materials (e.g., oils or fats) having a second multiplicity of molecular weights or viscosities, wherein said second mixture of fatty materials being external to or outside of said core/shell capsules; and (c) one or more surfactants, wherein the first mixture and the second mixture may be same or different.

The “insect” against which formulations of the invention are effective include flying and non-flying insects. The insects may be selected amongst insects that feed on fruits, leaves, stems, roots, and flowers of plants. These insects may include snails, leaf beetles, flea beetles, caterpillars, weevils, grasshoppers, katydids, ants, leaf cutter bees, beetles, flies, sawflies, moths, mealybugs, mites, spiders, borers (such as longhorned beetles, metallic wood boring beetles, engraver beetles, clearwing moths, American plum borer) and others.

In some embodiments, the insect is an arthropod. In some embodiments, the insect is a non-insect arthropod.

The surface is any flat or inclined surface of an object or a plant which is to be maintained permanently or over a period of time insect free. The surface may be one which is frequently visited by insects, or which insect incidental visit is to be avoided or prevented. In some embodiments, the surface is a surface of an object. In other embodiments, the surface is a surface of a living plant.

In some embodiments, the surface is a climbable surface, typically an inclined surface. The climbable surface may be a surface of an object, such as a wall (internal or external wall), a fence, a greenhouse walls or surfaces, planting pots, flower boxes and others; or of a plant, such as a tree trunk or a stem or a leaf of a living plant.

In some embodiments, the climbable surface is a tree trunk or a plant stem. The tree may be a deciduous tree, an evergreen, a fruit tree or a coniferous tree. Non-limiting examples of trees include avocado, citrus, mango, cacao, coffee etc.

In some embodiments, the plant is an ornamental plant or a garden plant.

In some embodiments, the plant is a fruit tree.

In some embodiment, the plant is of a height between 5 and 100 cm, or between 10 and 15 cm, or between 20 and 50 cm, or between 30 and 40 cm, or between 30 and 60 cm, or between 10 and 80 cm or between 50 and 100 cm.

The coating or film formed on the surface or a surface region may be formed by known means of application, such as by brushing, wiping, spraying, washing, dipping, smearing etc. Irrespective of the way by which the coating or film is formed, sufficient amount of a formulation of the invention is required to achieve a coating or film thickness that can be effective over long period of time. Typically, a thick coating or film is not required. However, the coating should be thick enough to achieve a uniform film or coat that is substantially free of surface features which would permit the insects to latch thereon. However, the film or coat need not be homogenous in thickness.

In some embodiments, the coating or film has a thickness of about 0.5 mm or smaller. In some embodiments, the thickness is between 5 and 200 μm (micron), or between 5 and 150 μm, or between 5 and 100 μm, or between 5 and 50 μm, or between 5 and 20 μm, or between 20 and 200 μm, or between 20 and 150, or between 20 and 100 μm, or between 20 and 50 μm, or between 50 and 200 μm, or between 50 and 150 μm, or between 50 and 100 μm, or between 100 and 200 μm, or between 100 and 150 μm, or between 150 and 200 μm, or is about 100 μm.

As used herein, the term “preventing” or any lingual variation thereof, encompasses the formulation or coating capabilities to minimize or diminish inset ability to latch on or interact with the surface to permit its movement, e.g., climbing, thereon. As tested, the preventive ability of formulations and coatings of the invention has been demonstrated over a period of several months. Generally, prevention was achieved with at least 90% efficacy over a period of at least 3 months.

In some embodiments, the preventive efficacy was between 90 and 99.99% over a period of 3 months, or between 90 and 99.99% over a period of between 2 and 4 months, or between 90 and 95% over a period of 2 to 4 months, or between 90 and 99.99% over a period of 1 to 6 months. The “efficiency” of prevention was determined by a variety of methods, such as that described in Example 5 (“Success rate (%)”).

The invention further provides a method of preventing insects, e.g., arthropods, from climbing on a climbable surface, the method comprising application of a formulations according to the invention onto said surface.

The invention further provides a kit, the kit comprising a formulation according to the invention and instructions of use.

In some embodiments, the capsules are provided dry and separately of the second mixture of fatty materials, and wherein the instructions provide a protocol for their combination. In some embodiments, the kit further comprises a receptacle for mixing the capsules and the second mixture.

The invention further provides a surfactant in a form of an adduct formed of poly (hydroxymethylsiloxane) (PHMS) and polyethylene glycol (PEG), wherein the PHMS and the PEG are associated to each other via covalent bonds (to provide a copolymer PHMS-g-PEG) or via hydrogen bonding.

In some embodiments, the surfactant is a copolymer of the form PHMS-g-PEG and represented by Formula (I):

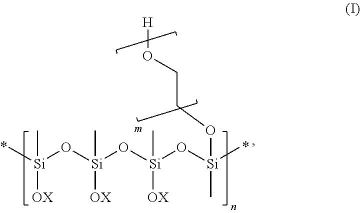

wherein X is H or PEG, m is an integer between 2-2,000; and n is an integer between 10-120. In compounds of Formula (I) wherein multiple PEGs are associated, each m may be same or different and selected between 2 and 2,000.

In some embodiments, the adduct is a product comprising PHMS and PEG, represented by Formula (II):

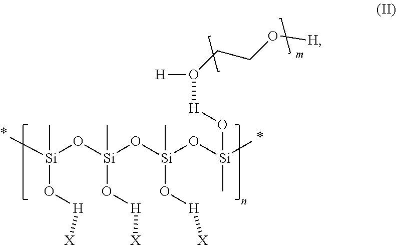

wherein X is absent or is PEG, m is an integer between 2-2,000; n is an integer between 10-120, and wherein “mmmmm” designates a hydrogen bond with X. In compounds of Formula (II) wherein multiple PEGs are associated, each m may be same or different and selected between 2 and 2,000.

As used herein, the term “adduct” is used to mean a product of association of PHMS and PEG, resulting in a single product containing both molecular entities. The interaction between the two molecular entities may be covalent or through H-bonding.

In a compound of formula (I) or (II), each of m may independently be an integer between 2 and 2,000, inclusive; or may be an integer between 2 and 20, or 2 and 50, or between 2 and 100, or between 2 and 200, or between 2 and 500, or between 2 and 1,000, or between 20 and 50, or between 20 and 100, or between 20 and 200, or between 20 and 500, or between 20 and 1,000, or between 20 and 2000, or between 50 and 100, or between 50 and 200, or between 50 and 500, or between 50 and 1,000, or between 50 and 2,000, or between 100 and 200, or between 100 and 500, or between 100 and 1,000, or between 100 and 2,000, or between 200 and 500, or between 200 and 1,000, or between 200 and 2,000, or between 500-1,000, or between 500 and 2,000, or between 1,000 and 2,000.

In some embodiments, in a compound of formula (I) or (II), each of n may independently of the other be an integer between 10 and 120, inclusive, or may be between 10 and 20, or between 10 and 50, or between 10 and 100, or between 20 and 50, or between 20 and 100, or between 20 and 120, or between 50 and 100, or between 50 and 120, or between 100 and 120.

The invention further provides a method of preparing the surfactant adduct, the method comprising:
treating PHMS in water under basic pH to obtain polyhydroxysiloxane (PHMS);
combining the basic water solution comprising PHMS with PEG, affording the PHMS/PEG adduct, as defined.

In some embodiments, the surfactant is a mixture of the adduct of formula (I) and (II).

In some embodiments, a basic pH is achievable by dissolving a base, such as KOH or NaOH in water.

In some embodiments, the PEG is PEG 400, PEG 6000, PEG 8000, or PEG 35000.

In some embodiments, the base: PHMS: PEG ratio (w/w/v) is 50-150:80-120:85-130. In some embodiments, the ratio is 120-130:90-110:105-115, or 125:100:110.

In some embodiments, the treating and/or combining is achievable at a temperature in the vicinity of room temperature, e.g., between 15 and 40° C. In some embodiments, the temperature is 20 and 30° C.

The invention further provides an object or an article of manufacture having at least a surface region thereof coated with a film that is not crossable by an insect.

In some embodiments, the film comprises a film-forming material, a mixture of fatty materials (e.g., oils or fats) having a multiplicity of molecular weights and/or viscosities, and optionally a surfactant.

In some embodiments, the film is formed by applying onto the surface region an aqueous formulation comprising at least one film forming material, a mixture of fatty materials (e.g., oils or fats) having a multiplicity of molecular weights or viscosities, and optionally a surfactant.

In some embodiments, the film forming material is provided in a form of capsules or carriers at least partially comprising said mixture of fatty materials and optionally the surfactant.

In some embodiments, the film forming material is provided in a form of capsules or carriers comprising at least a portion of said mixture of fatty materials and optionally the surfactant, wherein the remaining of said mixture and optionally the surfactant are present external to said capsules. Thus, the film may comprise (a) a plurality of capsules comprising said mixture of fatty materials and optionally the surfactant; and (b) a mixture of the fatty materials and optionally the surfactant outside of the capsules.

In some embodiments, the film may comprise two or more different populations of fatty materials, each being distinguishable in composition and/or amount (concentration, material ratios, etc) and/or form of presentation (i.e., loaded in a carrier or capsule or freely distributed in the formulation).

In some embodiments, the capsules are selected from core/shell capsules, microcapsules, microcarriers, and others.

In some embodiments, the film comprises (a) a plurality of core/shell capsules formed of a first film-forming material, wherein the core of said capsules comprising or being formed of a first mixture of fatty materials (e.g., oils or fats) having a first multiplicity of molecular weights or viscosities; (b) a mixture of a second film-forming material and a second mixture of fatty materials (e.g., oils or fats) having a second multiplicity of molecular weights or viscosities, wherein said second mixture of fatty materials being external to or outside of said core/shell capsules; and (c) one or more surfactants, wherein the first mixture and the second mixture may be same or different.

The invention further provides:

A film-forming formulation for preventing insects from crossing or climbing the surface, the formulation comprising at least one film forming material, a mixture of fatty materials having a multiplicity of molecular weights and/or viscosities, and optionally a surfactant.

In some configurations, the formulation is an aqueous formulation.

In some configurations, the at least one film forming material is provided in a form of capsules, wherein the capsules at least partially comprise said mixture of fatty materials and optionally the surfactant.

In some configurations, the at least one film forming material is provided in a form of capsules comprising at least a portion of said mixture of fatty materials and optionally the surfactant, and wherein a remaining of said mixture of fatty materials and optionally the surfactant is provided external to said capsules.

In some configurations, the formulation comprising (a) a plurality of capsules formed of the at least one film-forming material and comprising said mixture of fatty materials and optionally the surfactant; and (b) a mixture of the fatty materials and optionally the surfactant outside of the capsules.

In some configurations, the formulation comprising two or more different populations of fatty materials, each being distinguishable in composition and/or amount and/or form of presentation.

In some configurations, the capsules are core/shell capsules.

In some configurations, the formulation comprising (a) a plurality of core/shell capsules formed of the first film-forming material, wherein the core of said capsules comprising or being formed of a first mixture of fatty materials having a first multiplicity of molecular weights and/or viscosities; (b) a mixture of a second film forming material and a second mixture of fatty materials having a second multiplicity of molecular weights and/or viscosities, wherein said second mixture of fatty materials being external to or outside of said core/shell capsules; and (c) one or more first surfactants, wherein the first mixture and the second mixture are same or different.

A formulation comprising (a) a plurality of core/shell capsules formed of a first film-forming material, wherein the core of said capsules comprising or being formed of a first mixture of fatty materials having a first multiplicity of molecular weights and/or viscosities; (b) a mixture of a second film-forming material and a second mixture of fatty materials having a second multiplicity of molecular weights and/or viscosities, wherein said second mixture of fatty materials being external to or outside of said core/shell capsules; and (c) one or more first surfactants, wherein the first mixture and the second mixture are same or different.

In some configurations, the first mixture and the second mixture are same.

In some configurations, the first mixture and the second mixture are different.

In some configurations, the fatty materials are characterized by a hydrophilic-lipophilic balance (HLB) of 5 or higher.

In some configurations, the fatty materials are selected from oils and fats.

In some configurations, the fatty materials are selected from silicone oils, fruit oils, vegetable oils, animal oils, fatty acids, fatty acid amides, petroleum oils, and mineral oils.

In some configurations, the silicone oil is polydimethylsiloxane (PDMS) oil or a silicon oil or different molecular weights or viscosities.

In some configurations, the vegetable oil is selected from palm oil, soybean oil, rapeseed oil, sunflower oil, peanut oil, cottonseed oil, palm kernel oil, canola oil, soya oil, coconut oil, olive oil, corn oil, and sesame oil.

In some configurations, the fruit oil is selected from almond oil, grapefruit oil, coconut oil, peach oil, lemon oil, orange oil, avocado oil, and olive oil.

In some configurations, the fatty acid is selected from caprylic acid, capric acid, lauric acid, myristic acid, palmitic acid, stearic acid, arachidic acid, myristoleic acid, palmitoleic acid, sapienic acid, oleic acid, elaidic acid, vaccenic acid, linoleic acid, linoelaidic acid, and α-linolenic acid.

In some configurations, the fatty acid amide is an amide of a fatty acid according to claim 18, or a fatty acid amide selected from erucamide, oleamide, ethylene bis-oleamide, stearyl erucamide, oleyl palmitamide or any combination thereof.

In some configurations, the fatty material is or comprises almond oil.

In some configurations, the first mixture and/or the second mixture comprise at least two fatty materials having different viscosities between 5 and 100,000 cp (measured at 25° C.).

In some configurations, the different fatty materials are selected from fatty materials having viscosities between 350-60,000 cp, 100-1,000 cp, 40,000-80,000 cp, 35-45 cp, or 5-80 cp.

In some configurations, the first mixture comprises an oil having a viscosity between 100-1000 cp and another oil of a viscosity between 40,000-80,000 cp.

In some configurations, the first and/or second mixtures comprise a first oil having a viscosity between 35-45 cp, a second oil having a viscosity between 5-80 cp and a third oil having a viscosity between 100-1000 cp.

In some configurations, one of the first and second mixtures comprise almond oil with a viscosity of 35-45 cp and the second of the first and second mixtures comprises a vegetable oil with a viscosity of between 5-80 cp.

In some configurations, the first mixture comprises one or more silicone oils, or almond oil, or one or more vegetable oil, or one or more fatty acid amide, or one or more synthetic oil, or one or more fatty acids or any combination thereof.

In some configurations, the first mixture comprises a combination of any two or more of silicone oils, almond oil, vegetable oil, fatty acid amide, synthetic oil, and fatty acids.

In some configurations, the formulation comprising at least one silicone oil.

In some configurations, the at least one film-forming material is provided in a form of capsules comprising one or more silicone oils.

In some configurations, the film forming material is selected from polyvinyl alcohol, carnauba wax and bee wax.

In some configurations, the formulation comprising one or more silicone oil having a viscosity between 100 and 1,000 cp and one or more other silicone oil having a viscosity between 40,000 and 80,000 cp.

In some configurations, the formulation comprising a mixture of almond oil and a vegetable oil.

In some configurations, the formulation comprising erucamide, oleamide, ethylene bis-oleamide, stearyl erucamide, and oleyl palmitamide.

In some configurations, the formulation comprising at least one surfactant.

In some configurations, the first mixture comprises a mixture of silicone oils, almond oil, a vegetable oil and a fatty acid amide.

In some configurations, the first mixture comprises a mixture of silicone oils, wherein at least one silicone oil having a viscosity between 100 and 1,000 cp and another silicone oil having a viscosity between 40,000 and 80,000 cp.

In some configurations, the first mixture comprises a silicone oil having a viscosity between 100 and 500 cp, or a viscosity between 100 and 250 cp, or a viscosity between 250 and 1,000 cp, or a viscosity between 250 and 500 cp, or a viscosity of 350 cp, or a viscosity of between 500 and 1,000 cp, or a viscosity between 40,000 and 70,000 cp, or a viscosity between 40,000 and 50,000 cp, or a viscosity between 50,000 and 80,000 cp, or a viscosity between 50,000 and 70,000 cp, or a viscosity between 70,000 and 80,000 cp, or a viscosity of 60,000 cp.

In some configurations, the second mixture comprises almond oil with a viscosity between 35 and 45 cp and/or a vegetable oil with a viscosity between 5 and 80 cp.

In some configurations, the second mixture comprises a synthetic oil and/or a silicone oil.

In some configurations, the second mixture comprise a fatty material selected from palmitic acid, stearic acid, oleic acid, linoleic acid, N-acylethanolamine, oleamide, stearamide, erucamide and any combination thereof.

In some configurations, the second mixture comprises one or more silicone oils, or almond oil, or one or more vegetable oil, or one or more fatty acid amide, or one or more synthetic oil, or one or more fatty acids or any combination thereof.

In some configurations, the second mixture comprises a mixture of silicone oil, almond oil, a vegetable oil and a fatty acid amide.

In some configurations, the second mixture comprises a mixture of silicone oils, wherein at least one silicone oil having a viscosity between 100 and 1,000 cp and another silicone oil having a viscosity between 40,000 and 80,000 cp.

In some configurations, the second mixture comprises a silicone oil having a viscosity between 100 and 500 cp, or a viscosity between 100 and 250 cp, or a viscosity between 250 and 1,000 cp, or a viscosity between 250 and 500 cp, or a viscosity of 350 cp, or a viscosity of between 500 and 1,000 cp, or a viscosity between 40,000 and 70,000 cp, or a viscosity between 40,000 and 50,000 cp, or a viscosity between 50,000 and 80,000 cp, or a viscosity between 50,000 and 70,000 cp, or a viscosity between 70,000 and 80,000 cp, or a viscosity of 60,000 cp.

In some configurations, the formulation comprising one or more surfactants.

In some configurations, the first and second mixtures comprise same surfactants.

In some configurations, the surfactant is selected from polyhydroxy methylhydrosiloxane-polyethylene glycol copolymer (PHMS-g-PEG), polyhydroxymethylhydrosiloxane (PHMS), polyethylene glycol (PEG), PDMS and polyether in decamethylcyclopentasiloxane, cyclopentasiloxane, PEG/PPG-18/18 Dimethicone, lauryl PEG/PPG-18/18 methicone, cetyl PEG-PPG-10/1 dimethicone, bis-PEG/PPG-16/16 PEG/PPG-16/16 dimethicone, caprylic/capric triglyceride, 1-butene-N-vinylpyrrolidone copolymer, 1-hexadecnyl-N-vinylpyrrolidone copolymer, or any combination thereof.

In some configurations, the surfactant is selected from PHMS-g-PEG, PHMS, PEG and a chemical adduct of PHMS and PEG.

In some configurations, the surfactant is an adduct of PHMS and PEG, wherein the adduct is of formula (I) or (II):

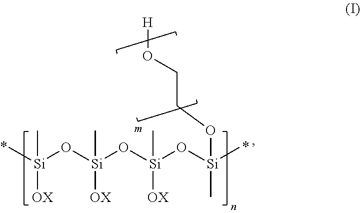

wherein X is H or PEG, m is an integer between 2 and 2,000; and n is an integer between 10-120;

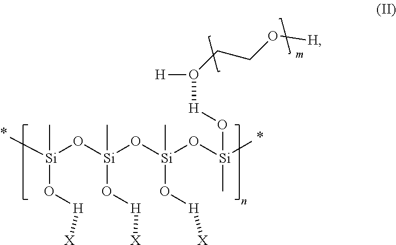

wherein X is absent or is PEG, m is an integer between 2 and 2,000; n is an integer between 10-120, and wherein “||||||||||||” designates a hydrogen bond with X.

In some configurations, the film forming material is a polymer or a wax material.

In some configurations, the film-forming material is selected from polyvinyl alcohol (PVOH), polyvinyl acetate (PVAc), polycaprolactone (PCL), a fatty alcohol, a biodegradable polyester, poly (furfuryl alcohol) bio-resin, a cellulosic material, lignin, a biobased wax, a paraffin wax, and any combination thereof.

In some configurations, the film-forming material is PVOH.

In some configurations, the surface is a plant surface or a surface of an object.

In some configurations, the surface is a climbable surface.

In some configurations, the insect is an arthropod or a non-insect arthropod.

A method of preventing insects from crossing or climbing a surface, the method comprising forming a coating or a film on said surface, wherein said coating or film comprises a film-forming material, a mixture of fatty materials having a multiplicity of molecular weights or viscosities, and optionally a surfactant.

In some configurations, the coating or film is formed by applying onto the surface a formulation according to the invention.

A method of preventing insects from climbing on a climbable surface, the method comprising application of a formulation according to the invention onto said surface.

A surfactant in a form of an adduct formed of poly (hydroxymethylsiloxane) (PHMS) and polyethylene glycol (PEG), wherein the PHMS and the PEG are associated to each other via covalent bonds or via hydrogen bonding.

In some configurations, the surfactant is a copolymer of the form PHMS-g-PEG represented by Formula (I):

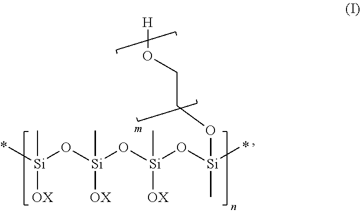

wherein X is H or PEG, m is an integer between 2 and 2,000; and n is an integer between 10-120.

In some configurations, the adduct is a product comprising PHMS and PEG, represented by Formula (II):

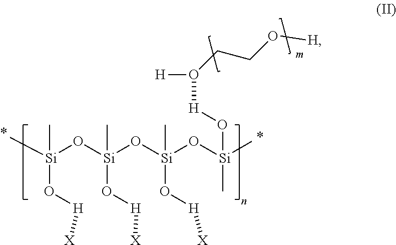

wherein X is absent or is PEG, m is an integer between 2-2,000; n is an integer between 10-120, and wherein “mmmmm” designates a hydrogen bond with X.

In some configurations, the surfactant is for use in a method of synthesis, method of film forming or in a method of formulation.

An object or an article of manufacture having at least a surface region thereof coated with a film comprising a film-forming material, a mixture of fatty materials having a multiplicity of molecular weights or viscosities, and optionally a surfactant.

In some configurations, the film is formed by applying onto the surface region a formulation according to the invention.

In some configurations, the surface is a wall (internal or external wall), a fence, a greenhouse walls or surfaces, planting pots, flower boxes and others; or a plant, a tree trunk, a stem, or a leaf.

In some configurations, the surface is an inclined surface selected from a wall (internal or external wall), a fence, a greenhouse walls or surfaces, planting pots, flower boxes and others; or a plant, a tree trunk, a stem, or a leaf.

In some configurations, the surface is a climbable surface selected from a tree trunk or a plant stem.

The object may be any structure, assembly, apparatus, plant, appliance or article of manufacture. In some embodiments, the object is an article of manufacture that can be appended or associated or placed on a surface to prevent crossing or climbing of insects.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1B depict ATR FTIR spectra of PHMS and PEG mixture before and after the reaction between both of them (see Examples 1-2). The bottom curve (having a peak at 2161 cm⁻¹) is the ATR absorption of the mixture of PEG and PHMS 1:1 before the reaction. The top curve is the ATR absorption of the PHMS-g-PEG after the reaction. FIG. 1A: ATR FTIR spectra of PHMS and PEG mixture before and after the reaction—full scale; and FIG. 1B: ATR FTIR spectra of PHMS and PEG mixture before and after the reaction—focus on ˜400-˜1400 cm⁻¹.

FIGS. 2A-2D depict experimental setups to assess the ability of ants to climb wood coated with the compositions of this invention. FIG. 2A: wood stick glued on a plastics lid; FIG. 2B: coated wood stick on it (the coating is White/Transparent). FIG. 2C: sticky tape with 100 μl of 50% Sucrose solution; and FIG. 2D: 10-15 Componotus ants added to the plastic container.

### DETAILED DESCRIPTION OF THE PRESENT INVENTION

In the following detailed description, numerous specific details are set forth in order to provide a thorough understanding of the invention. However, it will be understood by those skilled in the art that this invention may be practiced without these specific details. In other instances, well-known methods, procedures, and components have not been described in detail so as not to obscure this invention.

### Method of Preventing Arthropods Climbing on a Climbable Surface

In some embodiments, this invention provides a method of preventing arthropods climbing on a climbable surface, comprising application of a self-lubricating composition on the climbable surface, wherein the composition comprises capsules; and wherein the capsules comprise a core comprising a first mixture of oils and fats having multiplicity of molecular weights; a shell; and a first surfactant. In one embodiment, the prevention of arthropods climbing on a climbable surface comprises disruption of the Van der Waals interaction between the arthropods' legs (via hairy pads thereof) and the climbable surface.

In one embodiment, the method of this invention is sustainable, green, ecological and/or non-lethal, due to the utilization of the green and ecological, self lubricating composition, as described hereinbelow. In one embodiment, the method disrupts the Van der Waals interaction between the arthropods' legs (via hairy pads thereof) and the climbable surface, thus preventing the arthropods from climbing; and therefore this method does not harm and/or does not kill the arthropods. Each possibility represents a separate embodiment of this invention.

In one embodiment, the climbable surface is any surface that can be coated; and which arthropods can climb on (said surface). In one other embodiment, the climbable surface is a tree trunk, an internal or external wall, a fence or a greenhouse. In another embodiment, the climbable surface is a tree trunk. In other embodiment, the species of the tree is Avocado, Citrus, Mango, Cacao, Coffee or any ornamental plant species or fruit tree species. In another embodiment, any tree trunk is suitable in the context of this invention as long as no “internal pathways” are present in said trunk so the arthropod would not have any chance to “bypass” the coated climbable surface in order to climb the trunk from e.g. the inside of it (for example in a case where the tree has a hole around the ground so the arthropod can get inside the tree and climb from there). In another embodiment, the tree trunk is in a height of 5-100 cm, 10-15 cm, 20-50 cm, 30-40 cm, 30-60 cm, 10-80 cm or 50-100 cm. In another embodiment, the climbable surface is an internal wall. In another embodiment, the climbable surface is an external wall. In another embodiment, the climbable surface is a fence. In another embodiment, the climbable surface is a greenhouse. In another embodiment, Each possibility represents a separate embodiment of this invention.

In one embodiment, the application of the self lubricating composition comprises coating of the climbable surface with the self lubricating composition.

In one embodiment, the coating of the self lubricating composition comprises spraying or brushing of the climbable surface with the self lubricating composition.

In one embodiment, the arthropods are selected from the non-limiting group consisting of: insects, ants, mealybugs, mites, snails or spiders.

In one embodiment, the prevention was at least 90% efficient for 3 months. In another embodiment, the prevention was 90-99.99% efficient for 3 months. In another embodiment, the prevention was 90-99.99% efficient for 2-4 months. In another embodiment, the prevention was 90-95% efficient for 2-4 months. In another embodiment, the prevention was 90-99.99% efficient for 1-6 months. In another embodiment, the prevention was 90-95% efficient for 1-6 months. In some embodiments, said “efficiency” of the prevention is measured via various methods, for example the one described in Example 5 (“Success rate (%)”). Each possibility represents a separate embodiment of this invention.

In some embodiments, the self lubricating composition layer found on top of the climbable surface, or the self lubricating coating on top of the climbable surface has a thickness of 5-200 μm (micron). In one embodiment, the thickness is 5-150 μm. In another embodiment, the thickness is 5-100 μm. In another embodiment, the thickness is 5-50 μm. In another embodiment, the thickness is 5-20 μm. In another embodiment, the thickness is 20-200 μm. In another embodiment, the thickness is 20-150. In another embodiment, the thickness is 20-100 μm. In another embodiment, the thickness is 20-50 μm. In another embodiment, the thickness is 50-200 μm. In another embodiment, the thickness is 50-150 μm. In another embodiment, the thickness is 50-100 μm. In another embodiment, the thickness is 100-200 μm. In another embodiment, the thickness is 100-150 μm. In another embodiment, the thickness is 150-200 μm. In another embodiment, the thickness is 100 μm. Each possibility represents a separate embodiment of this invention.

### Self-Lubricating Composition and Capsule

In some embodiments, this invention provides a self-lubricating composition comprising capsules, wherein the capsules comprise a core comprising a first mixture of oils and fats having multiplicity of molecular weights; a shell; and a first surfactant. In one embodiment, the composition is an emulsion. In one other embodiment, the core of the capsule is an emulsion. In another embodiment, the emulsion of the composition and/or capsule is an oil in water or water in oil emulsion. In some embodiments, it is noted herein that the molecular weights are proportional to viscosities, thus said multiplicity of molecular weights comprise of and can be characterized by multiplicity of viscosities. Each possibility represents a separate embodiment of this invention.

In one embodiment, the shell comprises polyvinyl alcohol, Polycaprolactone, Fatty alcohol, biodegradable polyesters, poly (furfuryl alcohol) bioresin, Cellulose, lignin, biobased wax, paraffin wax, or any combination thereof. In another embodiment, the biobased wax comprises carnauba wax, bees wax, shellfish wax, lanolin, candelilla wax, jojoba oil, soy wax or any combination thereof. In another embodiment, the shell comprises at least one of polyvinyl alcohol, carnauba wax and bees wax in a weight ratio of 0-3:0-3:0-3. In another embodiment, the shell comprises at least one of polyvinyl alcohol, carnauba wax and bees wax in a weight ratio of 0-2:0-2:0-2. In another embodiment, the shell comprises at least one of polyvinyl alcohol, carnauba wax and bees wax in a weight ratio of 0-1:0-1:0-1. Each possibility represents a separate embodiment of this invention.

In one embodiment, the first mixture of oils and fats having multiplicity of molecular weights comprises a first mixture of silicone oils; and at least one of first almond oil; first vegetable oil; and fatty acid amide. In another embodiment, the first mixture of silicone oils comprises a first silicone oil having a viscosity of between 100-1,000 cp and a second silicone oil having a viscosity of between 40,000-80,000 cp. In another embodiment, the first silicone oil has a viscosity of between 100-500 cp. In another embodiment, the first silicone oil has between 100-250 cp. In another embodiment, the first silicone oil has a viscosity of between 250-1000 cp. In another embodiment, the first silicone oil has a viscosity of between 250-500 cp. In another embodiment, the first silicone oil has a viscosity of 350 cp. In another embodiment, the first silicone oil has a viscosity of between 500-1000 cp. In another embodiment, the second silicone oil has a viscosity of between 40,000-70,000 cp. In another embodiment, the second silicone oil has a viscosity of between 40,000-50,000 cp. In another embodiment, the second silicone oil has a viscosity of between 50,000-80,000 cp. In another embodiment, the second silicone oil has a viscosity of between 50,000-70,000 cp. In another embodiment, the second silicone oil has a viscosity of between 70,000-80,000 cp. In another embodiment, the second silicone oil has a viscosity of 60,000 cp. In another embodiment, the first mixture of silicone oils comprises a first silicone oil having a viscosity of 350 cp and a second silicone oil having a viscosity of 60,000 cp. In another embodiment, the first to second silicon oil weight ratio is 1-3:0-1. In another embodiment, the first to second silicon oil weight ratio is 1-2:0-1. In another embodiment, the first to second silicon oil weight ratio is 0.5-5:0-2. In another embodiment, the first silicone oil and the second silicone oil have a weight ratio of 3:1, respectively. In another embodiment, the first almond oil has a viscosity of 35-45 cp. In another embodiment, the first vegetable oil has a viscosity of 5-80 cp. In another embodiment, the first vegetable oil has a viscosity of 10-70 cp. In another embodiment, the first vegetable oil has a viscosity of 20-60 cp. In another embodiment, the first vegetable oil has a viscosity of 30-50 cp. In another embodiment, the first vegetable oil has a viscosity of 40-60 cp. Each possibility represents a separate embodiment of this invention.

In one embodiment, the fatty acid amide comprises crucamide, oleamide, ethylene bis-oleamide, stearyl erucamide, oleyl palmitamide or any combination thereof. In another embodiment, the fatty acid amide comprises a mixture of erucamide, oleamide, ethylene bis-oleamide, stearyl crucamide and oleyl palmitamide. Each possibility represents a separate embodiment of this invention.

In one embodiment, the capsules further comprise water. In other embodiment, the water are distilled water.

In some other embodiments, the composition further comprises a second surfactant and a second mixture of oils and fats having multiplicity of molecular weights. In one embodiment, the second mixture of oils and fats comprises a second mixture of silicone oils; and at least one of second almond oil; and second vegetable oil. In another embodiment, the second mixture of silicone oils comprise a third silicone oil having a viscosity of between 100-1,000 cp and a fourth silicone oil having a viscosity of between 40,000-80,000 cp. In another embodiment, the third silicone oil has a viscosity of between 100-500 cp. In another embodiment, the third silicone oil has a viscosity of between 100-250 cp. In another embodiment, the third silicone oil has a viscosity of between 250-1000 cp. In another embodiment, the third silicone oil has a viscosity of between 250-500 cp. In another embodiment, the third silicone oil has a viscosity of 350 cp. In another embodiment, the third silicone oil has a viscosity of between 500-1000 cp. In another embodiment, the fourth silicone oil has a viscosity of between 40,000-70,000 cp. In another embodiment, the fourth silicone oil has a viscosity of between 40,000-50,000 cp. In another embodiment, the fourth silicone oil has a viscosity of between 50,000-80,000 cp. In another embodiment, the fourth silicone oil has a viscosity of between 50,000-70,000 cp. In another embodiment, the fourth silicone oil has a viscosity of between 70,000-80,000 cp. In another embodiment, the fourth silicone oil has a viscosity of 60,000 cp. In another embodiment, the second mixture of silicone oils comprises a third silicone oil having a viscosity of 350 cp and a fourth silicone oil having a viscosity of 60,000 cp. In another embodiment, the third to fourth silicon oil weight ratio is 1-3:0-1. In another embodiment, the third to fourth silicon oil weight ratio is 1-2:0-1. In another embodiment, the third to fourth silicon oil weight ratio is 0.5-5:0-2. In another embodiment, the third silicone oil and the fourth silicone oil have a weight ratio of 3:1, respectively. In another embodiment, the second almond oil has a viscosity of 35-45 cp. In another embodiment, the second vegetable oil has a viscosity of 5-80 cp. In another embodiment, the second vegetable oil has a viscosity of 10-70 cp. In another embodiment, the second vegetable oil has a viscosity of 20-60 cp. In another embodiment, the second vegetable oil has a viscosity of 30-50 cp. In another embodiment, the second vegetable oil has a viscosity of 40-60 cp. Each possibility represents a separate embodiment of this invention.

In one embodiment, the composition (including, inter-alia, or excluding the capsules within) further comprises water.

In some embodiments, the first and the second surfactants are identical or different. In one embodiment, the first and second surfactants are different. In one other embodiment, the first and second surfactants are each independently polyhydroxymethylsiloxane-polyethylene glycol copolymer (PHMS-g-PEG), PHMS, PEG, Dow corning 5225c, Dow corning 5200, RITASIL sw 3050, ABIL care 85, Agrimer AL 10LC, Agrimer AL 22, Cyclopentasiloxane (and) PEG/PPG-18/18 Dimethicone, LAURYL PEG/PPG-18/18 METHICONE, Cetyl PEG-PPG-10/1 Dimethicone, Bis-PEG/PPG-16/16 PEG/PPG-16/16 Dimethicone (and) Caprylic/Capric Triglyceride, 1-Butene-N-vinylpyrrolidone copolymer, 1-hexadecnyl-N-vinylpyrrolidone copolymer, or any combination thereof. In one other embodiment, the first and second surfactants are each independently a polyhydroxymethylsiloxane-polyethylene glycol copolymer (PHMS-g-PEG). In one other embodiment, the first and surfactants are second each independently polyhydroxymethylsiloxane-polyethylene glycol copolymer (PHMS-g-PEG), PHMS, PEG or any combination thereof. In one other embodiment, the first and second surfactants are each independently a combination of PHMS and PEG. In one other embodiment, the first and second surfactants are each independently a combination of PHMS-g-PEG), PHMS and PEG. In yet one further embodiment, the first and second surfactants are each independently a combination of PHMS-g-PEG, PHMS and PEG, wherein the PHMS-g-PEG is 10-100, 10-50, 50-100, 10-20, 20-30, 30-40, 40-50, 50-60, 60-70, 70-80, 80-90 or 90-100% w/w of the whole combination. In yet other embodiment, the first and second surfactants are each independently Dow corning 5225c, Dow corning 5200, RITASIL sw 3050, ABIL care 85, Agrimer AL 10LC, Agrimer AL 22, Cyclopentasiloxane (and) PEG/PPG-18/18 Dimethicone, LAURYL PEG/PPG-18/18 METHICONE, Cetyl PEG-PPG-10/1 Dimethicone, Bis-PEG/PPG-16/16 PEG/PPG-16/16 Dimethicone (and) Caprylic/Capric Triglyceride, 1-Butene-N-vinylpyrrolidone copolymer, 1-hexadecnyl-N-vinylpyrrolidone copolymer, or any combination thereof. In other embodiment, the first and the second surfactants are useful in preventing emulsion, dispersion or suspension to break/collapse, i.e. they keep the stability of the composition and/or the capsules of this inventions. Each possibility represents a separate embodiment of this invention.

In some embodiments, the first and the second mixtures of oils and fats having multiplicity of molecular weights are identical or different. In one embodiment, the first and the second mixtures of oils and fats having multiplicity of molecular weights are different. In one other embodiment, the first and the second mixtures of oils and fats having multiplicity of molecular weights are identical. In yet other embodiment, each of the first and the second mixtures of oils and fats having multiplicity of molecular weights independently comprise two, three, four, five or more fatty/oily ingredients with specific (different) molecular weights and/or viscosities (proportional to molecular weights). In another embodiment, the first and/or the second mixtures of oils and fats having multiplicity of molecular weights comprise two fatty/oily ingredients with specific (different) molecular weights and/or viscosities. In another embodiment, the first and/or the second mixtures of oils and fats having multiplicity of molecular weights comprise three fatty/oily ingredients with specific (different) molecular weights and/or viscosities. In another embodiment, the first and/or the second mixtures of oils and fats having multiplicity of molecular weights comprise four fatty/oily ingredients with specific (different) molecular weights and/or viscosities. In another embodiment, the first and/or the second mixtures of oils and fats having multiplicity of molecular weights comprise five fatty/oily ingredients with specific (different) molecular weights and/or viscosities. In another embodiment, each ingredient within the multiple fatty/oily ingredients can be part of the same or a different family of oils/fats. In one another embodiment, non-limiting examples of oils/fats families consist essentially of: silicone oils, almond oils, vegetable oils, fatty acid amides, synthetic oils and fatty acids. Each possibility represents a separate embodiment of this invention.

In some embodiments, the first and the second almond oil are each independently selected from a bitter and a sweet almond oil and any combination thereof. In one embodiment, the first and/or second almond oil has each independently a viscosity of 35-45 cp. Each possibility represents a separate embodiment of this invention.

In some embodiments, the first and the second vegetable oil are each independently selected from olive oil, canola oil, corn oil, cotton seed oil, soya oil, coconut oil, and any combination thereof. In another embodiment, the first and/or second vegetable oil has each independently a viscosity of 5-80 cp. In another embodiment, the first and/or second vegetable oil has each independently a viscosity of 10-70 cp. In another embodiment, the first and/or second vegetable oil has each independently a viscosity of 20-60 cp. In another embodiment, the first and/or second vegetable oil has each independently a viscosity of 30-50 cp. In another embodiment, the first and/or second vegetable oil has a viscosity of 40-60 cp. In another embodiment, the olive oil has a viscosity of 40-60 cp. In another embodiment, the olive oil has a viscosity of 45-55 cp. Each possibility represents a separate embodiment of this invention.

In one embodiment, the capsule further comprises first synthetic oil and/or first fatty acid. In another embodiment, the first synthetic oil comprises polyalphaolefin (PAO) base oils, paraffin oil, polyalkylene glycol or any combination thereof. In another embodiment, the first synthetic oil replaces the first mixture of silicone oils. In yet another embodiment, the first synthetic oil is provided in addition to the first mixture of silicone oils. In another embodiment, the first fatty acid comprise palmitic acid, stearic acid, oleic acid, linoleic acid, N-acylethanolamine, oleamide, stearamide, crucamide or any combination thereof. In another embodiment, the first fatty acid replace the fatty acid amide. In yet another embodiment, the first fatty acid is provided in addition to the fatty acid amide. In one other embodiment, the first mixture of oils and fats having multiplicity of molecular weights of the capsule comprises the first mixture of silicone oils, first almond oil, first vegetable oil, fatty acid amide, first synthetic oil, first fatty acid or any combination thereof. Each possibility represents a separate embodiment of this invention.

In one embodiment, the composition further comprises second synthetic oil and/or second fatty acid. In another embodiment, the second synthetic oil comprises polyalphaolefin (PAO) base oils, paraffin oil, polyalkylene glycol or any combination thereof. In another embodiment, the second synthetic oil replaces the second mixture of silicone oils. In yet another embodiment, the second synthetic oil is provided in addition to the second mixture of silicone oils. In another embodiment, the second fatty acid comprise palmitic acid, stearic acid, oleic acid, linoleic acid, N-acylethanolamine, oleamide, stearamide, crucamide or any combination thereof. In one other embodiment, the second mixture of oils and fats having multiplicity of molecular weights of the composition comprises the second mixture of silicone oils, second almond oil, second vegetable oil, fatty acid amide, second synthetic oil, second fatty acid or any combination thereof. Each possibility represents a separate embodiment of this invention.

In some embodiments, the capsules comprise shell, first mixture of oils and fats having multiplicity of molecular weights, first surfactant and water. In one embodiment, the first mixture of oils and fats having multiplicity of molecular weights comprises a first mixture of silicone oils; and at least one of first almond oil; first vegetable oil; and fatty acid amide. In some embodiments, the capsules comprise shell, first mixture of silicone oils, first almond oil; first vegetable oil or mixture thereof; fatty acid amide; first surfactant and water. In some other embodiment, the capsules comprise 30-50% w/w of shell, 5-40% of w/w first mixture of silicone oils, 0-10% w/w of first almond oil; first vegetable oil or mixture thereof; 0-5% w/w of fatty acid amide; 1-5% w/w of first surfactant and 5-30% w/w water. In another embodiment, the shell is in a weight of 30-50% w/w, 30-40% w/w or 40-50% w/w. In another embodiment, the first mixture of silicone oils is in a weight of 5-40% w/w, 5-10% w/w, 10-20% w/w. 20-30% w/w or 30-40% w/w. In another embodiment, the first almond oil; first vegetable oil or mixture thereof is in a weight of 5-10% w/w. 0-5% w/w or 5-10% w/w. In another embodiment, the water is in a weight of 5-30% w/w, 5-10% w/w, 10-20% w/w or 20-30% w/w. In one embodiment, the capsules comprise at least one of polyvinyl alcohol, carnauba wax and bees wax, a first mixture of first silicone oil having a viscosity of between 100-1,000 cp and a second silicone oil having between a viscosity of 40,000-80,000 cp, mixture of first almond oil and first vegetable oil; crucamide, oleamide, ethylene bis-oleamide, stearyl crucamide, and oleyl palmitamide; PHMS-g-PEG, PHMS, PEG or any combination thereof; and water. In one other embodiment, the capsules comprise at least one of polyvinyl alcohol, carnauba wax and bees wax in a weight ratio of 0-3:0-3:0-3, a first mixture of first silicone oil having between 100-1,000 cp and a second silicone oil having a viscosity of between 40,000-80,000 cp, mixture of first almond oil and first vegetable oil; crucamide, oleamide, ethylene bis-oleamide, stearyl crucamide, and oleyl palmitamide; PHMS-g-PEG, PHMS, PEG or any combination thereof; and water. In one embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the shell is in a 40% w/w; and the first mixture of silicone oils comprises a first silicone oil having a viscosity of 350 cp and a second silicone oil having a viscosity of 60,000 cp. Each possibility represents a separate embodiment of this invention.

In some embodiments, the composition comprises capsules, a second surfactant, a second mixture of oils and fats having multiplicity of molecular weights and water. In one embodiment, the second mixture of oils and fats having multiplicity of molecular weights comprises a second mixture of silicone oils; and at least one of second almond oil; and second vegetable oil. In some embodiments, the composition comprises capsules, second mixture of silicone oils, mixture of second almond oil and second vegetable oil, second surfactant and water. In some other embodiments, the composition comprises 5-30% w/w capsules, 5-40% w/w second mixture of silicone oils, 0-15% w/w of mixture of second almond oil and second vegetable oil, 1-5% w/w of second surfactant and 10-50% w/w water. In one embodiment, the capsules are in a weight of 5-30% w/w, 5-10% w/w, 10-20% w/w or 20-30% w/w. In another embodiment, the second mixture of silicone oils is in a weight of 5-40% w/w, 5-10% w/w, 10-20% w/w 20-30% w/w or 30-40% w/w. In another embodiment, the mixture of second almond oil and second vegetable oil is in a weight of 0-15% w/w, 5-10% w/w or 10-15% w/w. In another embodiment, the water is in a weight of 0-50% w/w. 0-10% w/w. 10-20% w/w. 20-30% w/w, 30-40% w/w or 40-50% w/w. In one embodiment, the composition comprises capsules, second mixture of thirdsilicone oil having a viscosity of between 100-1,000 cp and a fourth silicone oil having a viscosity of between 40,000-80,000 cp, mixture of second almond oil and second vegetable oil; PHMS-g-PEG, PHMS, PEG or any combination thereof; and water. In one other embodiment, the composition comprises 5-30% w/w capsules, 5-40% w/w second mixture of thirdsilicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of thirdsilicone oil having a viscosity of 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. Each possibility represents a separate embodiment of this invention.

In some embodiments, the capsules are excluded from the composition of this invention. In one embodiment, the composition comprises a second surfactant and a second mixture of oils and fats having multiplicity of molecular weights, as indicated and detailed throughout this subject application; and said composition does not comprise the capsules as described throughout said subject application. In some other embodiments, the composition comprises a second surfactant, a second mixture of oils and fats having multiplicity of molecular weights and water. In one embodiment, the second mixture of oils and fats having multiplicity of molecular weights comprises a second mixture of silicone oils; and at least one of second almond oil; and second vegetable oil. In some other embodiments, the composition comprises second mixture of silicone oils, mixture of second almond oil and second vegetable oil, second surfactant and water. In some other embodiments, the composition comprises 15-50% w/w second mixture of silicone oils, 0-25% w/w of mixture of second almond oil and second vegetable oil, 1-5% w/w of second surfactant and 10-50% w/w water. In another embodiment, the second mixture of silicone oils is in a weight of 15-50% w/w, 15-20% w/w, 20-30% w/w, 30-40% w/w or 40-50% w/w. In another embodiment, the mixture of second almond oil and second vegetable oil is in a weight of 0-25% w/w, 0-5% w/w, 5-10% w/w, 10-15% w/w. 15-20% w/w or 20-25% w/w. In another embodiment, the water is in a weight of 0-50% w/w, 0-10% w/w, 10-20% w/w, 20-30% w/w. 30-40% w/w or 40-50% w/w. In one embodiment, the composition comprise second mixture of third silicone oil having a viscosity of between 100-1,000 cp and a fourth silicone oil having a viscosity of between 40,000-80,000 cp, mixture of second almond oil and second vegetable oil; PHMS-g-PEG, PHMS, PEG or any combination thereof; and water. In one other embodiment, the composition comprises 15-50% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having 350 cp and a fourth silicone oil having a viscosity of 60,000 cp. 0-25% w/w of mixture of second almond oil and second vegetable oil, 1-5% w/w of PHMS-g-PEG, PHMS, PEG or any combination thereof; and 10-50% w/w water. Each possibility represents a separate embodiment of this invention.

In some embodiments, the compositions that do include said capsules provide lower viscosity compared to compositions with the capsules absent, thus such capsules-including compositions are easier to spray on the climbable surface.

In some other embodiments, the capsule is a microcapsule. In one embodiment, the size of the microcapsule is 1-1000 μm (micron). In another embodiment, the size of the microcapsule is 1-500 μm. In another embodiment, the size of the microcapsule is 1-100 μm. In another embodiment, the size of the microcapsule is 1-50 μm. In another embodiment, the size of the microcapsule is 1-10 μm. In another embodiment, the size of the microcapsule is 1-5 μm. In another embodiment, the size of the microcapsule is 10-1000 μm. In another embodiment, the size of the microcapsule is 10-500 μm. In another embodiment, the size of the microcapsule is 10-100 μm. In another embodiment, the size of the microcapsule is 10-50 μm. In another embodiment, the size of the microcapsule is 50-1000 μm. In another embodiment, the size of the microcapsule is 50-500 μm. In another embodiment, the size of the microcapsule is 50-100 μm. In another embodiment, the size of the microcapsule is 100-1000 μm. In another embodiment, the size of the microcapsule is 100-500 μm. In another embodiment, the size of the microcapsule is 500-1000 μm. Each possibility represents a separate embodiment of this invention.

In one embodiment, the capsule is provided as detailed hereinabove in all embodiments and limitations, however in this specific embodiment the capsule is provided as a separate, distinct and independent embodiment/entity (not as a part of-, or within the-composition of this invention).

In some embodiments, the self-lubricating composition provides constant lubrication to the (climbable) surface upon which it is found or coated on, due to the multiplicity of the molecular weights (or viscosities, which are proportional to the molecular weights) of the oils and the fats which are released/diffused from said coating in a rate directly proportional to their weight/viscosity. Thus, multiple molecular weights provide multiple release/diffusion rates so the coating has lubrication for short range (of time; with low molecular weight oil/fat), medium and long range (with medium/high molecular weight oil/fat). The higher the multiplicity, the better the efficiency and/or the time said coating will stay efficient on the surface, though at least two molecular weights at the extremes (high and low) are sufficient as well. In one embodiment, the first mixture of oils within the capsule comprises a first mixture of silicone oils; comprising silicone oil of 100-1000 cp and another silicone oil of 40,000-80,000 cp; at least one of first almond oil of 35-45 cp; first vegetable oil of 5-80 cp; and a fatty acid amide (usually a solid fatty substance), and said first mixture of oils provides good lubrication and efficiency over time—90% over 3 months. In one embodiment, the second mixture of oils within the compositions comprises a second mixture of silicone oils; comprising silicone oil of 100-1000 cp and another silicone oil of 40,000-80,000 cp; and at least one of second almond oil of 35-45 cp; and second vegetable oil of 5-80 cp, and said second mixture of oils provides good lubrication and efficiency over time—90% over 3 months. In another embodiment, the first and second mixtures of oils provide together good lubrication and efficiency over time—90% over 3 months. In another embodiment, silicone oils mixture (e.g. as found within the first and/or second mixture of oils), for example, comprising silicone oil of 350 cp and another silicone oil of 60,000 cp—provides good lubrication and efficiency over time—90% over 3 months. In some other embodiments, the oils mixtures (e.g. first or second) provide fats/oils having a multiplicity of molecular weights or viscosities, for example three types such as low (around tens cp), medium (around hundreds cp) and high (around tens of thousands cp) viscosities, all together giving good lubrication and efficiency over time as described herein. In some further embodiments, each of the viscosity range/type (low, medium and high) comprises one or more type of oil or fat. In yet other embodiments, solid fats (e.g. fatty acid amide)/oils/waxes enhance said lubrication and efficiency over time, in addition to the oils mixtures having multiplicity of viscosities. In some other embodiments, the term “around” in the context of “around N cp” where Nis e.g. “tens” (or “hundreds” or “thousands”) is defined as the numerical range of (10−99)±5% (that is, the actual range of 10−99, and 10−5% (=“ten minus 5% of ten”) and also 99+5% (=“99 plus 5% of 99”)). Each possibility represents a separate embodiment of this invention.

In some embodiments, the composition is sustainable, green, ecological and/or non-lethal due to the ingredients involved within, as described hereinabove. In one embodiment, the method of this invention is also sustainable, green, ecological and/or non-lethal, due to the utilization of said composition. In one embodiment, the method disrupts the Van der Waals interaction between the arthropods' legs (via hairy pads thereof) and the climbable surface, thus preventing the arthropods climbing; and therefore this method does not harm and/or does not kill the arthropods. Each possibility represents a separate embodiment of this invention.

In some embodiments, the compositions as described herein, throughout all this subject application, and capsules within, and any embodiment and limitation associated therewith—can be used in the methods of this invention, as described hereinabove. Each such possibility, limitation or alternative of the composition is a separate embodiment of the composition and/or method of this invention.

In some embodiments, this invention provides a method of preparing the capsules, comprising:
stirring all ingredients to obtain the capsules; and
subjecting the capsule in a cold temperature to afford the capsules of the invention.

In some embodiments, this invention provides a method of preparing the compositions, comprising:
optionally, filtering the capsules of this invention (depending on whether capsules are used or not in the compositions); and
stirring all ingredients to obtain the compositions.

In one embodiment, the obtained capsules are microcapsules as described hereinabove.

In one embodiment, the stirring is performed for a duration of 0.1-10 hr. In another embodiment, the duration is 0.1-1 hr. In another embodiment, the duration is 0.1-2 hr. In another embodiment, the duration is 0.1-5 hr. In another embodiment, the duration is 1-2 hr. In another embodiment, the duration is 1-5 hr. In another embodiment, the duration is 1-10 hr. In another embodiment, the duration is 2-5 hr. In another embodiment, the duration is 5-10 hr. Each possibility represents a separate embodiment of this invention.

In one embodiment, the stirring is performed at 50-100° C. In another embodiment, the stirring is performed at 50-75° C. In another embodiment, the stirring is performed at 75-100° C. In another embodiment, the stirring is performed at 85° C. Each possibility represents a separate embodiment of this invention.

In one embodiment, the stirring is done in a high shear mixer (homogenizer).

In one embodiment, the emulsion is subjected to “cold temperature”, herein defined as −15° C.-5° C. In another embodiment, the cold temperature is 0° C.

In one embodiment, the size of capsules does not change once they are subjected to the “cold temperature” as defined hereinabove.

In some embodiments, the obtained capsules contain an emulsion, encapsulated by a shell.

### PHMS-PEG Copolymer

In some embodiments, this invention provides a copolymer PHMS-g-PEG. In one embodiment, the copolymer is represented by Formula (I):

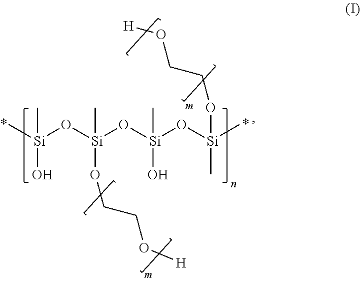

wherein
m is an integer between 2-2000; and
n is an integer between 10-120.

In one embodiment, m is an integer between 2-2000. In another embodiment, m is between 2-20. In another embodiment, m is between 2-50. In another embodiment, m is between 2-100. In another embodiment, m is between 2-200. In another embodiment, m is between 2-500. In another embodiment, m is between 2-1000. In another embodiment, m is between 2-2000. In another embodiment, m is between 20-50. In another embodiment, m is between 20-100. In another embodiment, m is between 20-200. In another embodiment, m is between 20-500. In another embodiment, m is between 20-1000. In another embodiment, m is between 20-2000. In another embodiment, m is between 50-100. In another embodiment, m is between 50-200. In another embodiment, m is between 50-500. In another embodiment, m is between 50-1000. In another embodiment, m is between 50-2000. In another embodiment, m is between 100-200. In another embodiment, m is between 100-500. In another embodiment, m is between 100-1000. In another embodiment, m is between 100-2000. In another embodiment, m is between 200-500. In another embodiment, m is between 200-1000. In another embodiment, m is between 200-2000. In another embodiment, m is between 500-1000. In another embodiment, m is between 500-2000. In another embodiment, m is between 1000-2000. Each possibility represents a separate embodiment of this invention.

In one embodiment, n is an integer between 10-120. In another embodiment, n is between 10-20. In another embodiment, n is between 10-50. In another embodiment, n is between 10-100. In another embodiment, n is between 20-50. In another embodiment, n is between 20-100. In another embodiment, n is between 20-120. In another embodiment, n is between 50-100. In another embodiment, n is between 50-120. In another embodiment, n is between 100-120. Each possibility represents a separate embodiment of this invention. In some embodiments, the PHMS-g-PEG copolymer is used as a surfactant. In one embodiment, the PHMS-g-PEG copolymer is used as a surfactant in the compositions and capsules of this invention as described hereinabove.

In some embodiments, this invention provides a method of preparing PHMS-g-PEG copolymer, comprising:
Dissolving a base in water;
Adding PHMS and water to the dissolved base until full dissolution;
Adding PEG (poly (ethylene glycol), affording the PHMS-g-PEG copolymer.

In some embodiments, the obtained PHMS-g-PEG copolymer is not pure and may include reactants such as PEG and/or PHMS. In one embodiment, the PHMS-g-PEG is 10-100, 10-50, 50-100, 10-20, 20-30, 30-40, 40-50, 50-60, 60-70, 70-80, 80-90 or 90-100% w/w of the whole combination (PHMS-g-PEG and reactants).

In one embodiment, the base is KOH, NaOH.

In one embodiment, the non-limiting examples of PEG consist of PEG 400, PEG 6000, PEG 8000, and PEG 35000.

In one embodiment, the Base: PHMS: PEG w/w/v ratio is 50-150:80-120:85-130. In another embodiment, the ratio is 120-130:90-110:105-115. In another embodiment, the ratio is 125:100:110. Each possibility represents a separate embodiment of this invention.

In one embodiment, the water is distilled water.

In one embodiment, the temperature within the method of preparing the copolymer is “room temperature”, defined herein as 15-40° C. In another embodiment, the temperature is 20-30° C.

In one embodiment, the duration of each step within the method of preparing the copolymer is 0.2-48 hr. In another embodiment, the duration is 0.2-1 hr. In another embodiment, the duration is 0.2-2 hr. In another embodiment, the duration is 0.2-5 hr. In another embodiment, the duration is 0.2-10 hr. In another embodiment, the duration is 0.2-24 hr. In another embodiment, the duration is 0.2-48 hr. In another embodiment, the duration is 1-2 hr. In another embodiment, the duration is 1-5 hr. In another embodiment, the duration is 1-10 hr. In another embodiment, the duration is 1-24 hr. In another embodiment, the duration is 1-48 hr. In another embodiment, the duration is 2-5 hr. In another embodiment, the duration is 2-10 hr. In another embodiment, the duration is 2-24 hr. In another embodiment, the duration is 2-48 hr. In another embodiment, the duration is 5-10 hr. In another embodiment, the duration is 5-24 hr. In another embodiment, the duration is 5-48 hr. In another embodiment, the duration is 10-24 hr. In another embodiment, the duration is 10-48 hr. In another embodiment, the duration is 24-48 hr. Each possibility represents a separate embodiment of this invention.

### Specific Capsules and Compositions

In some embodiments, the capsules comprise 30-50% w/w of shell, 5-40% of w/w first mixture of silicone oils, 0-10% w/w of first almond oil; first vegetable oil or mixture thereof; 0-5% w/w of fatty acid amide; 1-5% w/w of first surfactant and 5-30% w/w water. In one embodiment, the capsules comprise 30-40% w/w of shell, 5-40% of w/w first mixture of silicone oils, 0-10% w/w of first almond oil; first vegetable oil or mixture thereof; 0-5% w/w of fatty acid amide; 1-5% w/w of first surfactant and 5-30% w/w water. In another embodiment, the capsules comprise 40-50% w/w of shell, 5-40% of w/w first mixture of silicone oils, 0-10% w/w of first almond oil; first vegetable oil or mixture thereof; 0-5% w/w of fatty acid amide; 1-5% w/w of first surfactant and 5-30% w/w water. In another embodiment, the capsules comprise 30-50% w/w of shell, 5-10% of w/w first mixture of silicone oils, 0-10% w/w of first almond oil; first vegetable oil or mixture thereof; 0-5% w/w of fatty acid amide; 1-5% w/w of first surfactant and 5-30% w/w water. In another embodiment, the capsules comprise 30-50% w/w of shell, 10-20% of w/w first mixture of silicone oils, 0-10% w/w of first almond oil; first vegetable oil or mixture thereof; 0-5% w/w of fatty acid amide; 1-5% w/w of first surfactant and 5-30% w/w water. In another embodiment, the capsules comprise 30-50% w/w of shell, 20-30% of w/w first mixture of silicone oils, 0-10% w/w of first almond oil; first vegetable oil or mixture thereof; 0-5% w/w of fatty acid amide; 1-5% w/w of first surfactant and 5-30% w/w water. In another embodiment, the capsules comprise 30-50% w/w of shell, 30-40% of w/w first mixture of silicone oils, 0-10% w/w of first almond oil; first vegetable oil or mixture thereof; 0-5% w/w of fatty acid amide; 1-5% w/w of first surfactant and 5-30% w/w water. In another embodiment, the capsules comprise 30-50% w/w of shell, 5-40% of w/w first mixture of silicone oils, 0-5% w/w of first almond oil; first vegetable oil or mixture thereof; 0-5% w/w of fatty acid amide; 1-5% w/w of first surfactant and 5-30% w/w water. In another embodiment, the capsules comprise 30-50% w/w of shell, 5-40% of w/w first mixture of silicone oils, 5-10% w/w of first almond oil; first vegetable oil or mixture thereof; 0-5% w/w of fatty acid amide; 1-5% w/w of first surfactant and 5-30% w/w water. In another embodiment, the capsules comprise 30-50% w/w of shell, 5-40% of w/w first mixture of silicone oils, 0-10% w/w of first almond oil; first vegetable oil or mixture thereof; 0-5% w/w of fatty acid amide; 1-5% w/w of first surfactant and 5-10% w/w water. In another embodiment, the capsules comprise 30-50% w/w of shell, 5-40% of w/w first mixture of silicone oils, 0-10% w/w of first almond oil; first vegetable oil or mixture thereof; 0-5% w/w of fatty acid amide; 1-5% w/w of first surfactant and 10-20% w/w water. In another embodiment, the capsules comprise 30-50% w/w of shell, 5-40% of w/w first mixture of silicone oils, 0-10% w/w of first almond oil; first vegetable oil or mixture thereof; 0-5% w/w of fatty acid amide; 1-5% w/w of first surfactant and 20-30% w/w water. In some other embodiments, in any one of the capsules described above the first almond oil has a viscosity of 35-45 cp. In some other embodiments, in any one of the capsules described above the first vegetable oil has a viscosity of 5-80 cp. Each possibility represents a separate embodiment of this invention.

In some embodiments, the capsules comprise 30-50% w/w of at least one of polyvinyl alcohol, carnauba wax and bees wax, 5-40% w/w of a first mixture of first silicone oil having a viscosity of between 100-1,000 cp and a second silicone oil having a viscosity of between 40,000-80,000 cp. 0-10% w/w of mixture of first almond oil and first vegetable oil; 0-5% w/w of erucamide, oleamide, ethylene bis-oleamide, stearyl crucamide, and oleyl palmitamide; 1-5% w/w of PHMS-g-PEG, PHMS, PEG or any combination thereof; and 5-30% w/w water. In one embodiment, the capsules comprise 30-40% w/w of at least one of polyvinyl alcohol, carnauba wax and bees wax, 5-40% w/w of a first mixture of first silicone oil having a viscosity of between 100-1,000 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 40-50% w/w of at least one of polyvinyl alcohol, carnauba wax and bees wax, 5-40% w/w of a first mixture of first silicone oil having between 100-1,000 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-10% w/w of a first mixture of first silicone oil having a viscosity of between 100-1,000 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 10-20% w/w of a first mixture of first silicone oil having between 100-1,000 cp and a second silicone oil having a viscosity of between 40,000-80,000 cp. 0-10% w/w of mixture of first almond oil and first vegetable oil; 0-5% w/w of erucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 20-30% w/w of a first mixture of first silicone oil having between 100-1,000 cp and a second silicone oil having a viscosity of between 40,000-80,000 cp, 0-10% w/w of mixture of first almond oil and first vegetable oil; 0-5% w/w of erucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 30-40% w/w of a first mixture of first silicone oil having a viscosity of between 100-1,000 cp and a second silicone oil having between 40,000-80,000 cp. 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having between 100-200 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having a viscosity of between 200-500 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having a viscosity of between 500-800 cp and a second silicone oil having a viscosity of between 40,000-80,000 cp. 0-10% w/w of mixture of first almond oil and first vegetable oil; 0-5% w/w of crucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having between 800-1,000 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having between 100-1,000 cp and a second silicone oil having a viscosity of between 40,000-50,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having between 100-1,000 cp and a second silicone oil having a viscosity of between 50,000-70,000 cp. 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having a viscosity of between 100-1,000 cp and a second silicone oil having between 70,000-80,000 cp, 0-5% w/w of mixture of first almond oil and first vegetable oil; 0-5% w/w of crucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having a viscosity of between 100-1,000 cp and a second silicone oil having a viscosity of between 50,000-70,000 cp, 5-10% w/w of mixture of first almond oil and first vegetable oil; 0-5% w/w of erucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax, 5-40% w/w of a first mixture of first silicone oil having a viscosity of between 100-1,000 cp and a second silicone oil having a viscosity of between 50,000-70,000 cp, 0-10% w/w of mixture of first almond oil and first vegetable oil; 0-5% w/w of erucamide, oleamide, ethylene bis-oleamide, stearyl crucamide, and oleyl palmitamide; 1-5% w/w of PHMS-g-PEG, PHMS, PEG or any combination thereof; and 5-10% w/w water. In another embodiment, the capsules comprise 30-50% w/w of at least one of polyvinyl alcohol, carnauba wax and bees wax, 5-40% w/w of a first mixture of first silicone oil having between 100-1,000 cp and a second silicone oil having a viscosity of between 50,000-70,000 cp, 10-20% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 20-30% w/w water. In another embodiment, the capsules comprise 30-50% w/w of at least one of polyvinyl alcohol, carnauba wax and bees wax, 5-40% w/w of a first mixture of first silicone oil having between 100-1,000 cp and a second silicone oil having a viscosity of between 50,000-70,000 cp, 0-10% w/w of mixture of first almond oil and first vegetable oil; 0-5% w/w of erucamide, oleamide, ethylene bis-oleamide, stearyl erucamide, and olcyl palmitamide; 1-5% w/w of PHMS-g-PEG, PHMS, PEG or any combination thereof; and 5-30% w/w water. In some other embodiments, in any one of the capsules described above the first almond oil has a viscosity of 35-45 cp. In some other embodiments, in any one of the capsules described above the first vegetable oil has a viscosity of 5-80 cp. Each possibility represents a separate embodiment of this invention.

In some embodiments, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In one embodiment, the capsules comprise 30-40% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 40,000-80,000 cp, 0-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 40-50% w/w of at least one of polyvinyl alcohol, carnauba wax and bees wax in a weight ratio of 0-3:0-3:0-3, 5-40% w/w of a first mixture of first silicone oil having between 100-1,000 cp and a second silicone oil having a viscosity of between 40,000-80,000 cp, 0-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-1:0-1:0-1, 5-40% w/w of a first mixture of first silicone oil having between 100-1,000 cp and a second silicone oil having a viscosity of between 40,000-80,000 cp. 0-10% w/w of mixture of first almond oil and first vegetable oil; 0-5% w/w of erucamide, oleamide, ethylene bis-olcamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 1-3:1-3:1-3, 5-40% w/w of a first mixture of first silicone oil having a viscosity of between 100-1,000 cp and a second silicone oil having a viscosity of between 40,000-80,000 cp. 0-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 2-3:2-3:2-3, 5-40% w/w of a first mixture of first silicone oil having between 100-1,000 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having between 100-200 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 200-500 cp and a second silicone oil having between 40,000-80,000 cp. 0-10% w/w of mixture of first almond oil and first vegetable oil; 0-5% w/w of erucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 500-800 cp and a second silicone oil having a viscosity of between 40,000-80,000 cp, 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 800-1,000 cp and a second silicone oil having a viscosity of between 40,000-80,000 cp, 0-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having between 100-1,000 cp and a second silicone oil having a viscosity of between 40,000-50,000 cp. 0-10% w/w of mixture of first almond oil and first vegetable oil; 0-5% w/w of erucamide, oleamide, ethylene bis-oleamide, stearyl erucamide, and oleyl palmitamide; 1-5% w/w PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 50,000-70,000 cp. 0-10% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having between 100-1,000 cp and a second silicone oil having a viscosity of between 70,000-80,000 cp, 0-10% w/w of mixture of first almond oil and first vegetable oil; 0-5% w/w of crucamide, oleamide, ethylene bis-oleamide, stearyl crucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 40,000-80,000 cp, 0-5% w/w of mixture of first almond oil and first vegetable oil; 0-5% w/w of crucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 40,000-80,000 cp, 5-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 5-30% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 40,000-80,000 cp, 0-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 5-10% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having between 100-1,000 cp and a second silicone oil having a viscosity of between 40,000-80,000 cp, 0-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 10-20% w/w water. In another embodiment, the capsules comprise 30-50% w/w of at least one of polyvinyl alcohol, carnauba wax and bees wax in a weight ratio of 0-3:0-3:0-3, 5-40% w/w of a first mixture of first silicone oil having a viscosity of between 100-1,000 cp and a second silicone oil having a viscosity of between 40,000-80,000 cp, 0-10% w/w of mixture of first almond oil and first vegetable oil; 0-5% w/w of erucamide, oleamide, ethylene bis-oleamide, stearyl erucamide, and oleyl palmitamide; 1-5% w/w of PHMS-g-PEG, PHMS, PEG or any combination thereof; and 20-30% w/w water. In some other embodiments, in any one of the capsules described above the first almond oil has a viscosity of 35-45 cp. In some other embodiments, in any one of the capsules described above the first vegetable oil has a viscosity of 5-80 cp. Each possibility represents a separate embodiment of this invention.

In some embodiments, the composition comprises 5-30% w/w capsules, 5-40% w/w second mixture of silicone oils, 0-15% w/w of mixture of second almond oil and second vegetable oil, 1-5% w/w of second surfactant and 10-50% w/w water. In one embodiment, the composition comprises 5-10% w/w capsules, 5-40% w/w second mixture of silicone oils, 0-15% w/w of mixture of second almond oil and second vegetable oil, 1-5% w/w of second surfactant and 10-50% w/w water. In another embodiment, the composition comprises 10-20% w/w capsules, 5-40% w/w second mixture of silicone oils, 0-15% w/w of mixture of second almond oil and second vegetable oil, 1-5% w/w of second surfactant and 10-50% w/w water. In another embodiment, the composition comprises 20-30% w/w capsules, 5-40% w/w second mixture of silicone oils, 0-15% w/w of mixture of second almond oil and second vegetable oil, 1-5% w/w of second surfactant and 10-50% w/w water. In another embodiment, the composition comprises 5-30% w/w capsules, 5-10% w/w second mixture of silicone oils, 0-15% w/w of mixture of second almond oil and second vegetable oil, 1-5% w/w of second surfactant and 10-50% w/w water. In another embodiment, the composition comprises 5-30% w/w capsules, 10-20% w/w second mixture of silicone oils, 0-15% w/w of mixture of second almond oil and second vegetable oil, 1-5% w/w of second surfactant and 10-50% w/w water. In another embodiment, the composition comprises 5-30% w/w capsules, 20-30% w/w second mixture of silicone oils, 0-15% w/w of mixture of second almond oil and second vegetable oil, 1-5% w/w of second surfactant and 10-50% w/w water. In another embodiment, the composition comprises 5-30% w/w capsules, 30-40% w/w second mixture of silicone oils, 0-15% w/w of mixture of second almond oil and second vegetable oil, 1-5% w/w of second surfactant and 10-50% w/w water. In another embodiment, the composition comprises 5-30% w/w capsules, 5-40% w/w second mixture of silicone oils, 0-5% w/w of mixture of second almond oil and second vegetable oil, 1-5% w/w of second surfactant and 10-50% w/w water. In another embodiment, the composition comprises 5-30% w/w capsules, 5-40% w/w second mixture of silicone oils, 5-10% w/w of mixture of second almond oil and second vegetable oil, 1-5% w/w of second surfactant and 10-50% w/w water. In another embodiment, the composition comprises 5-30% w/w capsules, 5-40% w/w second mixture of silicone oils, 10-15% w/w of mixture of second almond oil and second vegetable oil, 1-5% w/w of second surfactant and 10-50% w/w water. In another embodiment, the composition comprises 5-30% w/w capsules, 5-40% w/w second mixture of silicone oils, 0-15% w/w of mixture of second almond oil and second vegetable oil, 1-5% w/w of second surfactant and 10-20% w/w water. In another embodiment, the composition comprises 5-30% w/w capsules, 5-40% w/w second mixture of silicone oils, 0-15% w/w of mixture of second almond oil and second vegetable oil, 1-5% w/w of second surfactant and 20-30% w/w water. In another embodiment, the composition comprises 5-30% w/w capsules, 5-40% w/w second mixture of silicone oils, 0-15% w/w of mixture of second almond oil and second vegetable oil, 1-5% w/w of second surfactant and 30-40% w/w water. In another embodiment, the composition comprises 5-30% w/w capsules, 5-40% w/w second mixture of silicone oils, 0-15% w/w of mixture of second almond oil and second vegetable oil, 1-5% w/w of second surfactant and 40-50% w/w water. In some other embodiments, in any one of the compositions described above the second almond oil has a viscosity of 35-45 cp. In some other embodiments, in any one of the compositions described above the second vegetable oil has a viscosity of 5-80 cp. Each possibility represents a separate embodiment of this invention.

In some embodiments, the composition comprises 5-30% w/w capsules, 5-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In one embodiment, the composition comprises 5-10% w/w capsules, 5-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 10-20% w/w capsules, 5-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 20-30% w/w capsules, 5-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-10% w/w second mixture of third silicone oil having a viscosity of between 100-1,000 cp and a fourth silicone oil having a viscosity of between 40,000-80,000 cp. 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 10-20% w/w second mixture of third silicone oil having a viscosity of between 100-1,000 cp and a fourth silicone oil having a viscosity of between 40,000-80,000 cp. 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 20-30% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 30-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-200 cp and a fourth silicone oil having a viscosity of between 40,000-80,000 cp. 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 200-500 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 500-800 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 800-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-1.000 cp and a fourth silicone oil having a viscosity of between 40,000-50,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-1,000 cp and a fourth silicone oil having a viscosity of between 50,000-70,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-1,000 cp and a fourth silicone oil having a viscosity of between 70,000-80,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-1,000 cp and a fourth silicone oil having a viscosity of between 40,000-80,000 cp. 0-5% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-1,000 cp and a fourth silicone oil having a viscosity of between 40,000-80,000 cp, 5-10% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-1,000 cp and a fourth silicone oil having a viscosity of between 40,000-80,000 cp, 10-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 10-20% w/w water. In another embodiment, the composition comprises 5-30% w/w capsules, 5-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 20-30% w/w water. In another embodiment, the composition comprises 5-30% w/w capsules, 5-40% w/w second mixture of third silicone oil having a viscosity of between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 30-40% w/w water. In another embodiment, the composition comprises 5-30% w/w capsules, 5-40% w/w second mixture of third silicone oil having between 100-1,000 cp and a fourth silicone oil having a viscosity of between 40,000-80,000 cp, 0-15% w/w of mixture of second almond oil and second vegetable oil, 1-5% w/w of PHMS-g-PEG, PHMS, PEG or any combination thereof; and 40-50% w/w water. In some other embodiments, in any one of the compositions described above the second almond oil has a viscosity of 35-45 cp. In some other embodiments, in any one of the compositions described above the second vegetable oil has a viscosity of 5-80 cp. Each possibility represents a separate embodiment of this invention.

In some embodiments, the composition comprises 5-30% w/w capsules, 5-40% w/w second mixture of third silicone oil having 350 cp and a second silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In one embodiment, the composition comprises 5-10% w/w capsules, 5-40% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 10-20% w/w capsules, 5-40% w/w second mixture of third silicone oil having 350 cp and a second silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 20-30% w/w capsules, 5-40% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-10% w/w second mixture of third silicone oil having 350 cp and a second silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 10-20% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 20-30% w/w second mixture of third silicone oil having 350 cp and a second silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 30-40% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having 350 cp and a fourth silicone oil having a viscosity of 60,000 cp, 0-5% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having 350 cp and a fourth silicone oil having a viscosity of 60,000 cp, 5-10% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having 350 cp and a fourth silicone oil having a viscosity of 60,000 cp, 10-15% w/w of mixture of second almond oil and second vegetable oil, 1-5% w/w of PHMS-g-PEG, PHMS, PEG or any combination thereof; and 10-50% w/w water. In another embodiment, the composition comprises 5-30% w/w capsules, 5-40% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 10-20% w/w water. In another embodiment, the composition comprises 5-30% w/w capsules, 5-40% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 20-30% w/w water. In another embodiment, the composition comprises 5-30% w/w capsules, 5-40% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 30-40% w/w water. In another embodiment, the composition comprises 5-30% w/w capsules, 5-40% w/w second mixture of third silicone oil having 350 cp and a fourth silicone oil having a viscosity of 60,000 cp, 0-15% w/w of mixture of second almond oil and second vegetable oil, 1-5% w/w of PHMS-g-PEG, PHMS, PEG or any combination thereof; and 40-50% w/w water. In some other embodiments, in any one of the compositions described above the second almond oil has a viscosity of 35-45 cp. In some other embodiments, in any one of the compositions described above the second vegetable oil has a viscosity of 5-80 cp. Each possibility represents a separate embodiment of this invention.

In some embodiments, the composition comprises 15-50% w/w second mixture of silicone oils, 0-25% w/w of mixture of second almond oil and second vegetable oil, 1-5% w/w of second surfactant and 10-50% w/w water. In another embodiment, the composition comprises 15-20% w/w second mixture of silicone oils, 0-25% w/w of mixture of second almond oil and second vegetable oil, 1-5% w/w of second surfactant and 10-50% w/w water. In another embodiment, the composition comprises 20-30% w/w second mixture of silicone oils, 0-25% w/w of mixture of second almond oil and second vegetable oil, 1-5% w/w of second surfactant and 10-50% w/w water. In another embodiment, the composition comprises 30-40% w/w second mixture of silicone oils, 0-25% w/w of mixture of second almond oil and second vegetable oil, 1-5% w/w of second surfactant and 10-50% w/w water. In another embodiment, the composition comprises 40-50% w/w second mixture of silicone oils, 0-25% w/w of mixture of second almond oil and second vegetable oil, 1-5% w/w of second surfactant and 10-50% w/w water. In another embodiment, the composition comprises 15-50% w/w second mixture of silicone oils, 0-5% w/w of mixture of second almond oil and second vegetable oil, 1-5% w/w of second surfactant and 10-50% w/w water. In another embodiment, the composition comprises 15-50% w/w second mixture of silicone oils, 5-10% w/w of mixture of second almond oil and second vegetable oil, 1-5% w/w of second surfactant and 10-50% w/w water. In another embodiment, the composition comprises 15-50% w/w second mixture of silicone oils, 10-15% w/w of mixture of second almond oil and second vegetable oil, 1-5% w/w of second surfactant and 10-50% w/w water. In another embodiment, the composition comprises 15-50% w/w second mixture of silicone oils, 15-20% w/w of mixture of second almond oil and second vegetable oil, 1-5% w/w of second surfactant and 10-50% w/w water. In another embodiment, the composition comprises 15-50% w/w second mixture of silicone oils, 20-25% w/w of mixture of second almond oil and second vegetable oil, 1-5% w/w of second surfactant and 10-50% w/w water. In another embodiment, the composition comprises 15-50% w/w second mixture of silicone oils, 0-25% w/w of mixture of second almond oil and second vegetable oil, 1-5% w/w of second surfactant and 10-20% w/w water. In another embodiment, the composition comprises 15-50% w/w second mixture of silicone oils, 0-25% w/w of mixture of second almond oil and second vegetable oil, 1-5% w/w of second surfactant and 20-30% w/w water. In another embodiment, the composition comprises 15-50% w/w second mixture of silicone oils, 0-25% w/w of mixture of second almond oil and second vegetable oil, 1-5% w/w of second surfactant and 30-40% w/w water. In another embodiment, the composition comprises 15-50% w/w second mixture of silicone oils, 0-25% w/w of mixture of second almond oil and second vegetable oil, 1-5% w/w of second surfactant and 40-50% w/w water. In some other embodiments, in any one of the compositions described above the second almond oil has a viscosity of 35-45 cp. In some other embodiments, in any one of the compositions described above the second vegetable oil has a viscosity of 5-80 cp. Each possibility represents a separate embodiment of this invention.

In some embodiments, the composition comprises 15-50% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-20% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 20-30% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 30-40% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 40-50% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 100-200 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having between 200-500 cp and a fourth silicone oil having a viscosity of between 40,000-80,000 cp. 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having between 500-800 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 800-1,000 cp and a fourth silicone oil having a viscosity of between 40,000-80,000 cp. 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 100-1,000 cp and a fourth silicone oil having a viscosity of between 40,000-50,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 100-1,000 cp and a fourth silicone oil having a viscosity of between 50,000-70,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 100-1,000 cp and a fourth silicone oil having a viscosity of between 70,000-80,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 100-1,000 cp and a fourth silicone oil having a viscosity of between 40,000-80,000 cp, 0-5% w/w of mixture of second almond oil and second vegetable oil, 1-5% w/w of PHMS-g-PEG, PHMS, PEG or any combination thereof; and 10-50% w/w water. a viscosity of In another embodiment, the composition comprises 15-50% w/w second mixture of third silicone oil having a viscosity of between 100-1,000 cp and a fourth silicone oil having between 40,000-80,000 cp. 10-15% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 100-1,000 cp and a fourth silicone oil having a viscosity of between 40,000-80,000 cp, 15-20% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having between 100-1,000 cp and a fourth silicone oil having a viscosity of between 40,000-80,000 cp, 20-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 10-20% w/w water. In another embodiment, the composition comprises 15-50% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 20-30% w/w water. In another embodiment, the composition comprises 15-50% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 30-40% w/w water. In another embodiment, the composition comprises 15-50% w/w second mixture of third silicone oil having a viscosity of between 100-1,000 cp and a fourth silicone oil having a viscosity of between 40,000-80,000 cp, 0-25% w/w of mixture of second almond oil and second vegetable oil, 1-5% w/w of PHMS-g-PEG, PHMS, PEG or any combination thereof; and 40-50% w/w water. In some other embodiments, in any one of the compositions described above the second almond oil has a viscosity of 35-45 cp. In some other embodiments, in any one of the compositions described above the second vegetable oil has a viscosity of 5-80 cp. Each possibility represents a separate embodiment of this invention.

In some embodiments, the composition comprises 15-50% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-20% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 20-30% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 30-40% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 40-50% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of 350 cp and a fourth silicone oil having a viscosity of 60,000 cp, 0-5% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of 350 cp and a fourth silicone oil having a viscosity of 60,000 cp. 5-10% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of 350 cp and a fourth silicone oil having a viscosity of 60,000 cp. 10-15% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of 350 cp and a fourth silicone oil having a viscosity of 60,000 cp, 15-20% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of 350 cp and a fourth silicone oil having a viscosity of 60,000 cp, 20-25% w/w of mixture of second almond oil and second vegetable oil, 1-5% w/w of PHMS-g-PEG, PHMS, PEG or any combination thereof; and 10-50% w/w water. In another embodiment, the composition comprises 15-50% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 10-20% w/w water. In another embodiment, the composition comprises 15-50% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 20-30% w/w water. In another embodiment, the composition comprises 15-50% w/w second mixture of third silicone oil having a viscosity of 350 cp and a fourth silicone oil having a viscosity of 60,000 cp. 0-25% w/w of mixture of second almond oil and second vegetable oil, 1-5% w/w of PHMS-g-PEG, PHMS, PEG or any combination thereof; and 30-40% w/w water. In another embodiment, the composition comprises 15-50% w/w second mixture of third silicone oil having a viscosity of 350 cp and a fourth silicone oil having a viscosity of 60,000 cp, 0-25% w/w of mixture of second almond oil and second vegetable oil, 1-5% w/w of PHMS-g-PEG, PHMS, PEG or any combination thereof; and 40-50% w/w water. Each possibility represents a separate embodiment of this invention.

### First and Second Compositions and Kits Thereof

In some embodiments, this invention provides a kit, comprising a first composition, a second composition, and instructions to use in a method of preventing arthropods from climbing on a climbable surface. In one embodiment, the first and second compositions are kept in separate containers which form part of the kit.

In some embodiments, this invention provides a first composition.

In some embodiments, this invention provides a second composition.

In some embodiments, the first composition serves as a mechanical barrier between the climbable surface and the second composition serves as the active layer in preventing arthropods from climbing. In one other embodiment, each of the compositions as described hereinabove (prior to “First and second compositions and kits thereof” section) can be used as the first or second composition. In another embodiment, the first composition comprises the shell as described hereinabove; the first silicone oil as described hereinabove, having a viscosity of between 100-1000 cp; the first surfactant as described hereinabove and water. In another embodiment, the first composition comprises the shell as described hereinabove, at 5-20% w/w; the first silicone oil as described hereinabove, having a viscosity of between 100-1000 cp, at 10-30% w/w; the first surfactant as described hereinabove, at 0.5-10% w/w; and water, at 30-80% w/w. In one other embodiment, the first composition comprises polyvinylalcohol, first silicone oil of 350 cp; PHMS-g-PEG, PHMS, PEG or any combination thereof; and water. In one other embodiment, the first composition comprises polyvinylalcohol at 5-20% w/w, first silicone oil of 350 cp at 10-30% w/w; PHMS-g-PEG, PHMS, PEG or any combination thereof at 0.5-10% w/w and water, at 30-80% w/w. In one other embodiment, the first composition comprises polyvinylalcohol at 12% w/w, first silicone oil of 350 cp at 20% w/w; PHMS-g-PEG, PHMS, PEG or any combination thereof at 4% w/w and water, at 64% w/w. Each possibility represents a separate embodiment of this invention. Each possibility (e.g. identity of components; and concentrations thereof and ranges of the same) and combination of the same, as described hereinabove (non-limiting examples are found for example in the “Specific capsules and compositions” section, however any possibility as found throughout this document is plausible)—represent a separate embodiment of this invention.

In another embodiment, the second composition comprises an aqueous phase and an oily phase. In another embodiment, the aqueous phase comprises the shell as described hereinabove and water. In another embodiment, the aqueous phase comprises the shell as described hereinabove, at 0.5-10% w/w; and water at 90-99.5% w/w. In another embodiment, the aqueous phase comprises polyvinylalcohol and water. In another embodiment, the aqueous phase comprises polyvinylalcohol at 0.5-10% w/w; and water at 90-99.5% w/w. In another embodiment, the aqueous phase comprises polyvinylalcohol at 5% w/w; and water at 95% w/w. Each possibility represents a separate embodiment of this invention. Each possibility (e.g. identity of components; and concentrations thereof and ranges of the same) and combination of the same, as described hereinabove (non-limiting examples are found for example in the “Specific capsules and compositions” section, however any possibility as found throughout this document is plausible)—represent a separate embodiment of this invention.

In another embodiment, the oily phase comprises the third silicone oil as described hereinabove, having a viscosity of between 100-1000 cp; the fourth silicone oil as described hereinabove, having a viscosity of between 40,000-80,000 cp; the first almond oil as described hereianbove; the first vegetable oil as described hereinabove; and the second surfactant as described hereinabove. In another embodiment, the oily phase comprises the third silicone oil as described hereinabove, having a viscosity of between 100-1000 cp, at 40-70% w/w; the fourth silicone oil as described hereinabove, having a viscosity of between 40,000-80,000 cp, at 10-30% w/w; the first almond oil as described hereinabove, at 1-15% w/w; the first vegetable oil as described hereinabove, at 1-15% w/w; and the second surfactant as described hereinabove, at 0.5-10% w/w. In another embodiment, the oily phase comprises the third silicone oil, having a viscosity of 350 cp; the fourth silicone oil, having a viscosity of between 60,000 cp; the first almond oil having molecular weight of 300 gr/mol; the first vegetable oil, having a molecular weight of 1520 gr/mol; and PHMS-g-PEG, PHMS, PEG or any combination thereof. In another embodiment, the oily phase comprises the third silicone oil, having a viscosity of 350 cp, at 40-70% w/w; the fourth silicone oil, having a viscosity of between 60,000 cp, at 10-30% w/w; the first almond oil having molecular weight of 300 gr/mol, at 1-15% w/w; the first vegetable oil, having a molecular weight of 1520 gr/mol, at 1-15% w/w; and PHMS-g-PEG, PHMS, PEG or any combination thereof, at 0.5-10% w/w. In another embodiment, the oily phase comprises the third silicone oil, having a viscosity of 350 cp, at 55% w/w; the fourth silicone oil, having a viscosity of between 60,000 cp, at 20% w/w; the first almond oil having molecular weight of 300 gr/mol, at 10% w/w; the first vegetable oil, having a molecular weight of 1520 gr/mol, at 10% w/w; and PHMS-g-PEG, PHMS, PEG or any combination thereof, at 5% w/w. In one embodiment, the vegetable oil is an olive oil. Each possibility represents a separate embodiment of this invention. Each possibility (e.g. identity of components; and concentrations thereof and ranges of the same) and combination of the same, as described hereinabove (non-limiting examples are found for example in the “Specific capsules and compositions” section, however any possibility as found throughout this document is plausible)—represent a separate embodiment of this invention.

In one further embodiment, this invention provides methods of preparing the first and second compositions of the kit. In one embodiment, the first composition is prepared in a method comprising mixing (between 1-240 minutes, e.g. 1, 2, 10, 20, 60, 120 or 240 minutes) and heating (to between 40-90° C., e/g/40,50, 60, 70, 80, 85 or 90° C.), optionally via homogenizer of the corresponding ingredients, which are detailed hereinabove. The aqueous and oily phase of the second compositions are prepared in a similar method to the first composition, each independently can be prepared by unique combination of heating temperature and time (of the corresponding ingredients). In one embodiment, the second composition is prepared by a method comprising mixing and heating together (as described hereinabove) the oily and aqueous phases. In another embodiment, the ratio of oily to aqueous phase is between 4:1 to 1:4; or 1:1; or 3:2; or 2:3. Each possibility represents a separate embodiment of this invention.

In some embodiments, this invention provides method of preventing arthropods climbing on a climbable surface, comprising application of the first composition of the kit on the climbable surface, wherein the climbable surface is as described hereinabove; followed by application of the second composition in the same. In some other embodiment, the obtained thickness of the layers/films is as the same as described hereinabove in the “Self-lubricating composition and capsule” section. In one embodiment, there should be between 30-120, 5-10, 20-30, 10-30, 30-60, 60-90 or 90-120 minutes between the applications of the first and second compositions. Each possibility represents a separate embodiment of this invention.

In some embodiments, the term “PHMS-g-PEG, PHMS, PEG or any combination thereof” or “PHMS-g-PEG, PHMS, PEG or any combination thereof”, as found throughout this whole document, is defined as only PHMS, only PEG, only PHMS-PEG or any possible combination between the triad, including e.g. PHMS-g-PEG, PHMS and PEG as one combination; and PHMS and PEG as other combination, amongst other combinations. In one other embodiment, the combination is PHMS-g-PEG, PHMS and PEG, wherein the PHMS-g-PEG is 10-100, 10-50, 50-100, 10-20, 20-30, 30-40, 40-50, 50-60, 60-70, 70-80, 80-90 or 90-100% w/w of the whole combination. Each possibility represents a separate embodiment of this invention.

In some embodiments, when a ratio of “X:0” (reading, “X:zero”, wherein X is an integer) is indicated in any one of this document's embodiments—the zero means a value of “around zero”, i.e. very close to zero (for example, milli (10⁻³), micro (10⁻⁶), nano (10⁻⁹), etc.). The following examples are presented in order to more fully illustrate the preferred embodiments of this invention. They should in no way, however, be construed as limiting the broad scope of this invention.

### EXAMPLES

### Example 1: Preparation of PHMS-g-PEG

125 gr KOH was dissolved in 50 ml distilled water, and to the mixture was dripped 100 gr PHMS cautiously and slowly. While adding PHMS, were added 350 ml of distilled water in portions, 100 ml each time, while maintaining the prevention of the exothermic reaction. After adding all the reagents, the mixture kept stirring overnight, all the solid phase was dissolved, and at room temp were added 110 ml of PEG 400, the reaction was carried out slowly and the color of the solution changed from colorless to yellow-orange. The obtained material was isolated in one of the following ways: a) the crude solution was dried until provision of a dried solid; or b) an acid was added to the crude solution to arrive at pH=7, where precipitation occurred and the precipitate was isolated and dried.

### Example 2: Characterization of the PHMS-g-PEG Copolymer Via ATR-FTIR

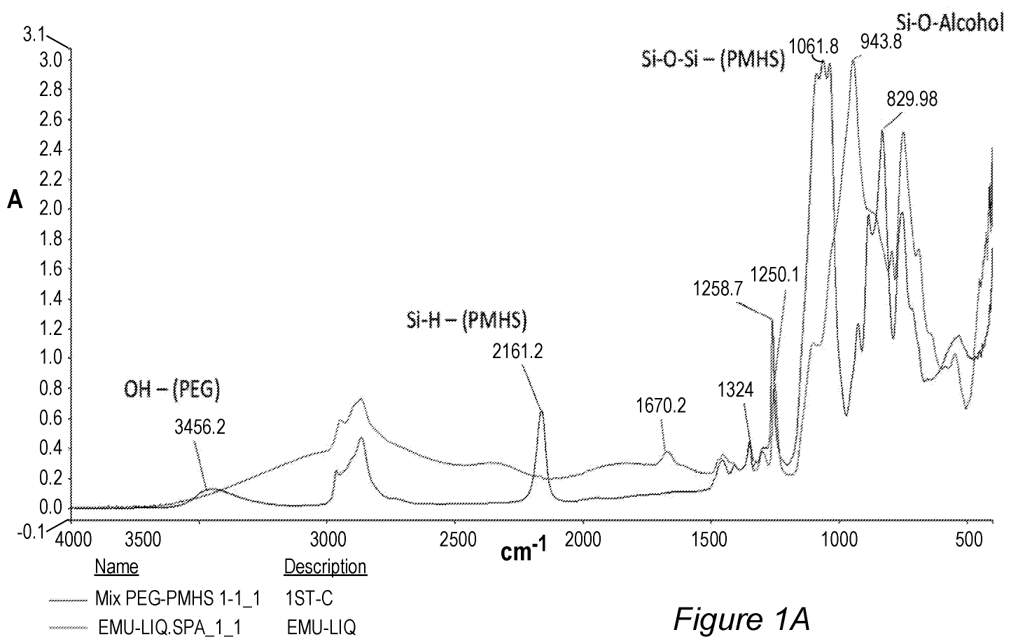

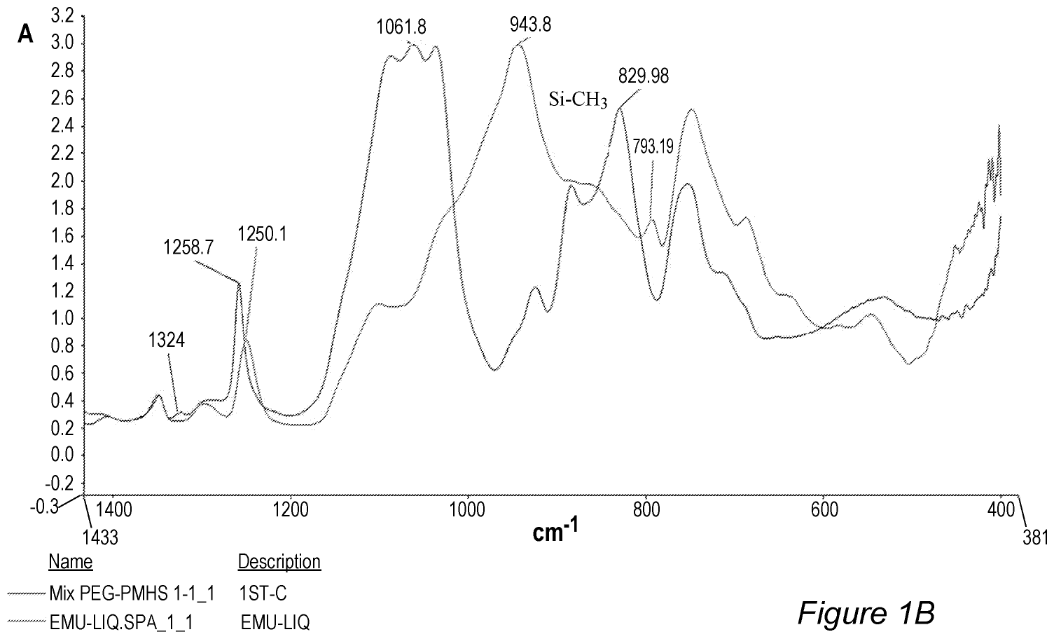

The PHMS-g-PEG prepared in Example 1 was characterized via ATR-FTIR. The bottom curve (having a peak at 2161 cm⁻¹) is the ATR absorption of the mixture of PEG and PHMS 1:1 before the reaction. The top curve is the ATR absorption of the PHMS-g-PEG after the reaction. FIGS. 1A-1B show that the PHMS-g-PEG is characterized by different features with respect to the mixture before the reaction (PEG: PHMS 1:1). The main differences are shown at 2161 cm⁻¹ and 944 cm⁻¹. Most of the Si—H groups (2161 cm 1) are consumed in PHMS-g-PEG, which is further confirmed by the Hydrogen that evolved during the reaction process. A new sharp peak at 944 cm⁻¹ is observed in PHMS-g-PEG that doesn't exist in the mixture before the reaction and it is characteristic to Si—O-Alcohol (Si—O-PEG).

### Example 3: Capsules for Self Lubricating Composition Comprising PHMS-g-PEG as Surfactant

Ingredients within the capsule (% are w/w):
40% polyvinylalcohol+Carnauba wax+Bee wax (0-3:0-3:0-3)
5-40% a mixture of Silicone oils (350 cp and 60000 cp) 1-3:0-1
0-10% almond oil/vegetable oils (<100 cp)
0-5% mixture of Fatty acid amides;
Erucamide, Olcamide, Ethylene bis-olcamide (EBO), Stearyl crucamide (SE), Oleyl palmitamide (OP)
1-5% surfactant (PHMS-g-PEG)
5-30% distilled water

All ingredients were stirred in a high-shear mixer (homogenizer) for 10 min at 85° C. When a homogeneous and uniform emulsion was obtained, the emulsion was placed into an ice bowl to freeze the size of the capsules. Microcapsules were obtained.

### Example 4: Self Lubricating Composition Comprising PHMS-g-PEG as Surfactant

Ingredients within the composition (% are w/w):
5-30% Microcapsules of Example 3
5-40% a mixture of Silicone oils (350 cp and 60000 cp) 3:1
0-15% almond oil/vegetable oils (<100 cp)
1-5% surfactant (PHMS-g-PEG)
10-50% distilled water

The microcapsules of Example 3 were filtered and added to an emulsion composition having the above ingredients in the concentration as indicated hereinabove. All ingredients were stirred in a high-shear mixer (homogenizer) for 10 min at room temperature.

### Example 5: Inhibition of Ants Climbing on a Wood Coated with the Self Lubricating Compositions of this Invention

Ants consume honeydew which is produced by homopterans (Aphids). By exploiting hemipteran honeydew, ants provide a sanitary service to the pests and protect them from parasitoids and other enemies. Ants also transport these hemipterans to safer sites where they can flourish. Thus, the relationship between sap-sucking homopterans and attending ants is mutually beneficial, but may be detrimental to the host plants, as expanding hemipteran colonies take more of their nutrients. (Martinez, JJ.I., Cohen, M. & Mgocheki, N. The response of an aphid tending ant to artificial extra-floral nectaries on different host plants. Arthropod-Plant Interactions 5, 185-192 (2011).

### Materials and Equipment

1. Componotus Ants
2. Wood sticks 10 cm×1 cm×0.2 cm
3. 100 ml transparent rounded plastic containers+lid
4. Yellow sticky insects tape
5. Sugar syrup concentration of 50%
6. self lubricating compositions, e.g. Example 4.

### Test Method

The experimental system consists of one common ant species, Componotus (Hymenoptera: Formicidae). The ants are collected from e.g. Al Qassimi college campus garden and maintained in big plastic containers with soil from their nest.

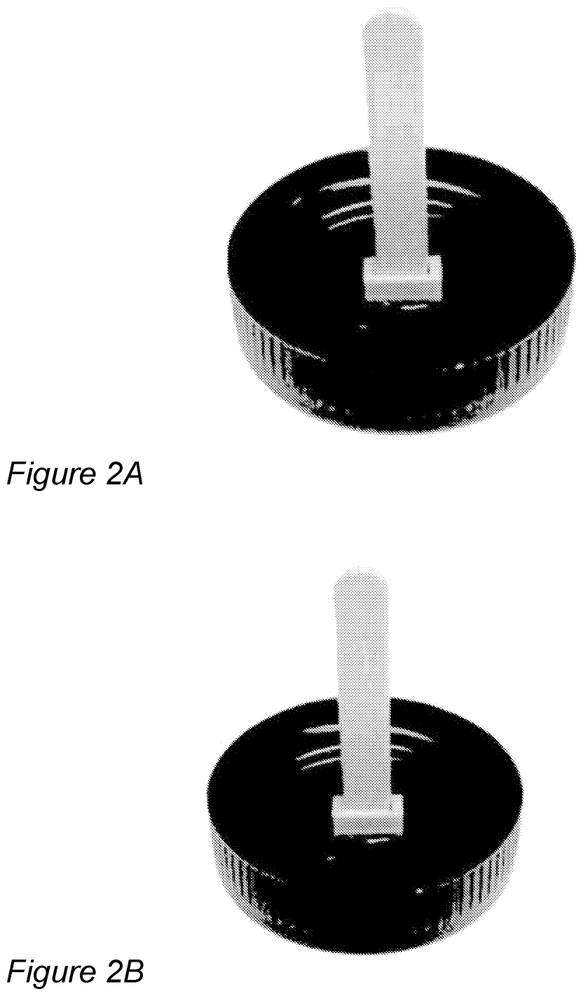

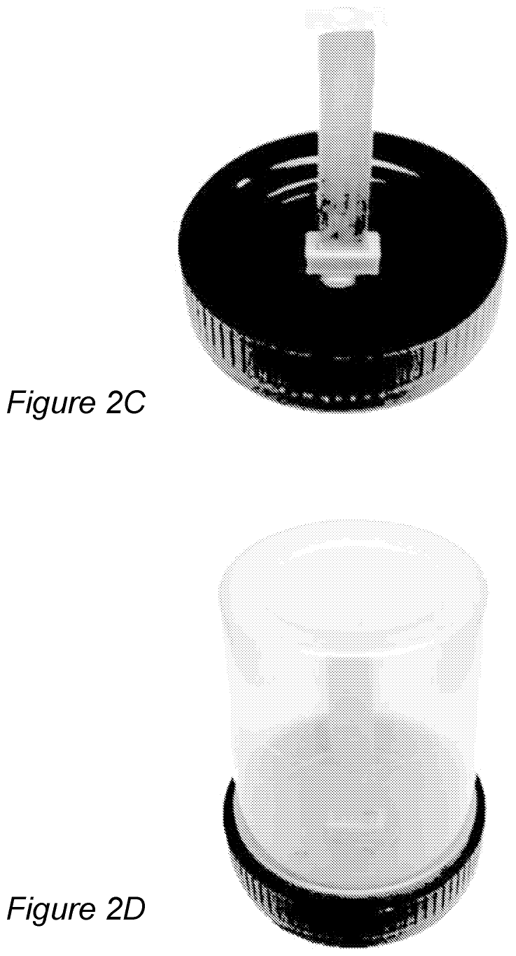

Success rate of ants climbing on the coated wood is measured by counting the number of ants that get stuck on a sticky trap on top of the wood stick, which was coated with the compositions beforehand. Rounded transparent plastic containers (100 ml) are used for each coating material. A wood stick (7.5 cm) is glued to each plastic container lid (FIG. 2A) to mimic a tree trunk. Every wood stick is sprayed with said compositions, e.g. Example 4, and remains on the table for 15-20 minutes to get dry (FIG. 2B). Small piece of sticky tape (0.75 cm*1 cm) is placed on top of each wood stick with a drop of 100 μl of 50% sucrose solution to mimic the honeydew that the aphids produce to attract ants (FIG. 2C).

Ten to fifteen Componotus ants that were subjected to a diet without food or water for 3 days prior to the experiment are placed in each plastic container (FIG. 2D). Each ant is not used more than one time to avoid any adaptive or learning effects. The experiment is replicated at least 6 times with each different composition and ant. The experiment is done in 12 hours light/12 hours dark rounds, 22-25° C. for 72 hours. After 72 hours, the number of stuck ants is collected.

The coating success rate is calculated as follows:

$$Success Rate ( \% ) = 100 - ( \frac{number of successful ants}{number of tested ants} \times 100 )$$

The insect is considered “successful” if it did pass the coating material and did reach the edge of the wood stick and got stuck on the yellow sticker. Success rates for the compositions of this invention are found to be high.

### Results

Coating of vertical wood surfaces with the self lubricating compositions of this invention (e.g. Example 4) results in failure of the Componotus ants to adhere to- and to climb on said vertical wood surfaces, thus their climbing on said surfaces is inhibited and/or prevented by these compositions.

### Example 6: Preparing the Two Layers/Compositions of the Kit of this Invention First Layer/Composition Ingredients (w/w)

64% water
12% polyvinylalcohol
20% Silicone oil 350 cp
4% PHMS-g-PEG

All ingredients were put in one vessel and then mixed (in a homogenizer) while heated to 85° C., for 10 minutes.

### Second Layer/Composition:

Aqueous Phase Ingredients (w/w):
95% water
5% polyvinylalcohol

The materials were dissolved and mixed at 85° C.

Oily Phase Ingredients (w/w):
55% silicone oil 350 cp
20% Silicone oil 60,000 cp
10% almond oil (300 gr/mol)
10% olive oil (1520 gr/mol)
5% PHMS-g-PEG

The oily phase was heated to 85° C., then it was added to the aqueous phase in a ratio of 3:2 (oily: aqueous), and finally all phases were mixed together at homogenizer for 10 minutes.

### Example 7: Coating with Two Layers/Compositions Kit

The kit included both first and second compositions in separate containers. The first composition of Example 6 is applied on a tree trunk at 10-15 cm height, providing a 100 micron thickness layer. Following 10-30 minutes from application of first layer, the second composition is applied on top of the first, providing 100 micron thickness of a second layer (same height as first layer). Then prevention of ants from climbing on said tree trunk is tested as detailed in e.g. Example 5. The test shows that the kit of said compositions is successful in inhibiting ants from climbing the tree trunk.

### Example 8: Microcapsule Preparation

### Oils Phase:

40% PVOH+Carnauba wax+Bee wax (0-3:0-3:0-3);
5-40% a mixture of Silicone oils (350 cp and 60000 cp) 1-3:0-1;
0-10% almond oil/vegetable oils;
0-5% mixture of Fatty acid amide; Erucamide, Oleamide, Ethylene bis-oleamide (EBO), Stearyl erucamide (SE), Oleyl palmitamide (OP);
1-5% surfactant (such as PHMS adduct);
5-30% distilled water.

All ingredients were stirred in a high-shear mixer (homogenizer) for 10 min at 85° C.

When a homogeneous and uniform emulsion is obtained, the emulsion was placed into an ice bowl to freeze the size of the microcapsules. The microcapsules were then filtered and added to an emulsion according to the following concentrations:
3-20% PVOH+Carnauba wax+Bee wax (0-3:0-3:0-3);
5-30% Microcapsules;
5-40% a mixture of Silicone oils (10 cp and 100000 cp) 3-1:1-;
0-15% almond oil/vegetable oils;
1-5% surfactant (PHMS g-PEG); and
10-50% distilled water.

While certain features of this invention have been illustrated and described herein, many modifications, substitutions, changes, and equivalents will now occur to those of ordinary skill in the art. It is, therefore, to be understood that the appended claims are intended to cover all such modifications and changes as fall within the true spirit of this invention.

1.-67. (canceled)

68. A film-forming formulation for preventing insects from crossing or climbing the surface, the formulation comprising at least one film forming material, a mixture of fatty materials having a multiplicity of molecular weights and/or viscosities, and optionally a surfactant, wherein the formulation is optionally an aqueous formulation.

69. The formulation according to claim 68, wherein the at least one film forming material is provided in a form of capsules, wherein the capsules at least partially comprise said mixture of fatty materials and optionally the surfactant.

70. The formulation according to claim 69, the formulation comprising (a) a plurality of core/shell capsules formed of the first film-forming material, wherein the core of said capsules comprising or being formed of a first mixture of fatty materials having a first multiplicity of molecular weights and/or viscosities; (b) a mixture of a second film forming material and a second mixture of fatty materials having a second multiplicity of molecular weights and/or viscosities, wherein said second mixture of fatty materials being external to or outside of said core/shell capsules; and (c) one or more first surfactants, wherein the first mixture and the second mixture are same or different.

71. The formulation according to claim 70, wherein the first mixture and the second mixture are same or different.

72. The formulation according to claim 68, wherein the fatty materials are selected from silicone oils, fruit oils, vegetable oils, animal oils, fatty acids, fatty acid amides, petroleum oils, and mineral oils.

73. The formulation according to claim 68, wherein the at least one film-forming material is provided in a form of capsules comprising one or more silicone oils.

74. The formulation according to claim 68, wherein the film forming material is selected from polyvinyl alcohol, carnauba wax and bee wax.

75. The formulation according to claim 73, comprising a mixture of almond oil and a vegetable oil.

76. The formulation according to claim 70, wherein the first mixture comprises a mixture of silicone oils, almond oil, a vegetable oil and a fatty acid amide.

77. The formulation according to claim 70, wherein the second mixture comprises a synthetic oil and/or a silicone oil.

78. The formulation according to claim 68, wherein the surfactant is selected from PHMS-g-PEG, PHMS, PEG and a chemical adduct of PHMS and PEG.

79. The formulation according to claim 78, wherein the surfactant is an adduct of PHMS and PEG, wherein the adduct is of formula (I) or (II): 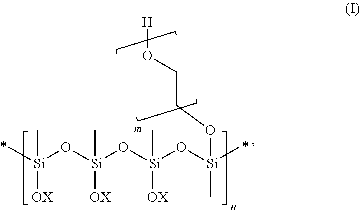
wherein X is H or PEG, m is an integer between 2 and 2,000; and n is an integer between 10-120; 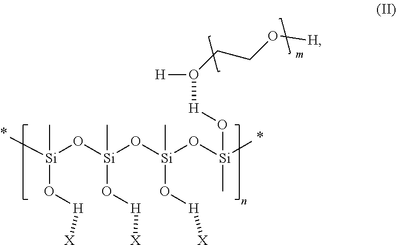
wherein X is absent or is PEG, m is an integer between 2 and 2,000; n is an integer between 10-120, and wherein “||||||||||||” designates a hydrogen bond with X.

80. The formulation according to claim 68, wherein the film forming material is a polymer or a wax material.

81. The formulation according to claim 68, wherein the insect is an arthropod or a non-insect arthropod.

82. A method of preventing insects from crossing or climbing a surface, the method comprising forming a coating or a film on said surface, wherein said coating or film comprises a film-forming material, a mixture of fatty materials having a multiplicity of molecular weights or viscosities, and optionally a surfactant.

83. A surfactant in a form of an adduct formed of poly (hydroxymethylsiloxane) (PHMS) and polyethylene glycol (PEG), wherein the PHMS and the PEG are associated to each other via covalent bonds or via hydrogen bonding.

84. The surfactant according to claim 83, being a copolymer of the form PHMS-g-PEG represented by Formula (I): 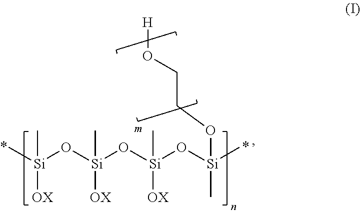
wherein X is H or PEG, m is an integer between 2 and 2,000; and n is an integer between 10-120.

85. The surfactant according to claim 83, wherein the adduct is a product comprising PHMS and PEG, represented by Formula (II): 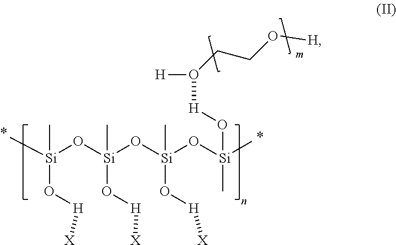
wherein X is absent or is PEG, m is an integer between 2-2,000; n is an integer between 10-120, and wherein “||||||||||||” designates a hydrogen bond with X.

86. An object or an article of manufacture having at least a surface region thereof coated with a film comprising a film-forming material, a mixture of fatty materials having a multiplicity of molecular weights or viscosities, and optionally a surfactant.

87. The object according to claim 86, being a wall (internal or external wall), a fence, a greenhouse walls or surfaces, planting pots, flower boxes and others; or a plant, a tree trunk, a stem, or a leaf.
